(19) United States
(12) Patent Application Publication
(10) Pub. No.: US 2010/0197643 A1
(43) Pub. Date: Aug. 5, 2010

(54) PHYTOCHEMICAL-RICH OILS AND METHODS RELATED THERETO

(76) Inventor: Stephen T. Talcott, College Station, TX (US)

Correspondence Address: MEDLEN & CARROLL, LLP, 101 HOWARD STREET, SUITE 350, SAN FRANCISCO, CA, 94105, US

(21) Appl. No.: 12/602,698

(22) PCT Filed: Jun. 12, 2008
(86) PCT No.: PCT/US08/07325

§ 371 Date: Apr. 22, 2010

Related U.S. Application Data
(60) Provisional application No. 60/934,763, filed on Jun. 15, 2007.

(51) Int. Cl. A61K 31/56; A61K 8/63; A61P 39/00; A61Q 90/00

(52) U.S. Cl. 514/171

(57) ABSTRACT

The invention relates to methods used in extracting oil from plants, plant fruits, and/or nuts, preferably fruits from plants of the family Arecaceae, or Palmae, even more preferably acai fruit. In some embodiments, the invention relates to methods of extracting the oil using an extraction solution comprising a volatile alcohol and volatile ketone. In other embodiments, the invention relates to oil from acai fruit and acai fruit by-products that contain enriched concentrations of phytochemicals.

### FIELD OF INVENTION

The invention relates to methods used in extracting oil from plants, plant fruits, and/or nuts, preferably fruits from plants of the family Arecaceae, or Palmae, even more preferably açai fruit. In some embodiments, the invention relates to methods of extracting the oil using an extraction solution comprising a volatile alcohol and/or volatile ketone. In other embodiments, the invention relates to oil from açai fruit and açai fruit by-products that contain enriched concentrations of phytochemcals.

### BACKGROUND

The Palm Family, Arecaceae; is a family of flowering plants belonging to the monocot order, Arecales. There are roughly 202 currently known genera with around 2600 species, most of which are restricted to tropical or subtropical climates. Most palms are distinguished by their large, compound, evergreen leaves arranged at the top of an unbranched stem. The fruit of many species of palm are rich in oils that contain beneficial phytochemicals that have antioxidant properties, such as polyphenolics, phytosterols, and mono- and polyunsaturated fatty acids. Thus, there is a need for improved methods for isolating such compositions.

### SUMMARY OF INVENTION

The invention relates to methods used in extracting oil from plants, plant fruits, and/or nuts, preferably fruits from plants of the family Arecaceae, or Palmae, even more preferably from plants of the genus Euterpe, including but not limited to the açai fruit (Euterpe oleracea or Euterpe precatoria). In some embodiments, the invention relates to methods of extracting the oil using an extraction solution comprising a volatile alcohol and volatile ketone. In other embodiments, the invention relates to oil from açai fruit and açcai fruit by-products that contain enriched concentrations of phytochemicals.

In some embodiments, the invention relates to an isolated non-naturally occurring oil composition comprising: greater than 50% by weight unsaturated fatty acids; greater than 10% by weight saturated fatty acids; and greater than 0.1% by weight and less than 0.5% and by weight and even more preferably less than 0.2% by weight of polyphenolics; and greater than 0.1% by weight and less than 0.5% by weight and even more preferably less than 0.2% of phytosterols. In further embodiments said oil comprises greater than 80% and even more preferably greater than 90% by weight triacylglycerols.

In some embodiments, the invention relates to an isolated non-naturally occurring oil composition comprising: greater than 50% by weight unsaturated fatty acids; greater than 10% by weight saturated fatty acids; and greater than 5%, 1%, 0.5, 0.15% or 0.10% or 0.05%, or 0.01% by weight of polyphenolics and greater than 2%, 1%, 0.5%, 0.2% , 0.1% or 0.05% or 0.01% by weight of phytosterols. In further embodiments, said polyphenolics comprise: protocatechuic acid (3,4-dihydroxybenzoic acid); procyanidin C1 (Epicatechin-(4beta-->8)epicatechin-(4beta-->8)epicatechin or dimmers, e.g., B1, B2, B3, and B4; p-hydroxybenzoic acid; (+)-catechin; vanillic acid; (−)-epicatechin; proanthocyanidin A2 ((+)-Epicatechin-(4beta-8,2beta-O-7)-epicatechin) or dimmers; and ferulic acid. In further embodiments, the oil comprises greater than 10 mg per liter of protocatechuic acid (3,4-dihydroxybenzoic acid). In further embodiments, the oil comprises greater than 10 mg per liter of vanillic acid. In further embodiments, the oil comprises beta-sitosterol, stigmasterol, and campesterol. In further embodiments, the oil composition is obtained from an açai fruit. In further embodiments, said saturated and unsaturated fatty acids do not contain trans fatty acids. In further embodiments, said composition is 60-90% by weight unsaturated fatty acids, 10-20% by weight saturated fatty acids, greater than 1%, 0.5%, 0.15% or 0.10% or 0.05%, or 0.01% by weight or between 5-15% polyphenolics, and greater than 1%, 0.5%, 0.2%, 0.1%, 0.05 or 0.01% by weight or between 2-10% phytosterols. In further embodiments, said composition comprises 60-80% by weight monounsaturated fatty acids, 10-20% by weight polyunsaturated fatty acids, and by weight 10-20% saturated fatty acids. In further embodiments, the composition comprises less than 1, 5, 10 or 20% by weight of amino acids. In further embodiments, said oil has a specific gravity of about 0.93. In further embodiments, said oil has a refractive index of about 1.47. In further embodiments, said açai oil contains a total polyphenolics concentration of at least 1, 5, 10, 100, 500, 1,000, 1,200, 1,400, 2,000, or 2,500 mg per liter of oil. In further embodiments, said açai oil contains a total phytosterol concentration of at least 1, 5, 10, 100, 500, 1,000, 1,200, 1,400, 2,000, or 2,500 mg per liter of oil. In further embodiments, the açai oil contains a total polyphenolics concentration of less than 1, 5, 10, 100, 500, 1,000, 1,200, 1,400, 2,000, or 2,500 mg per liter of oil. In further embodiments, said açai oil contains a total phytosterol concentration of less than 1, 5, 10, 100, 500, 1,000, 1,200, 1,400, 2,000, or 2,500 mg per liter of oil.

In some embodiments, the invention relates to a method of isolating an oil comprising: a) providing a composition comprising a portion of a plant, plant fruit, or nut, preferably a palm fruit; b) mixing said composition with an extraction solution comprising an alcohol and a ketone such that a second solution comprising a set of insoluble components is formed; c) filtering said second solutions to separate said set of insoluble components providing a third solution; and d) isolating an oil by removing volatile components from said third solution. Although this method is preferred for isolating oils from plant of the Arecaceae family, even more preferably açai fruit and composition comprising portions of açai fruit, such as the skin, pulp, and nut, it is contemplated that this method could be used to isolate from any variety of constituents with desired hydrophobicity from plants and portions thereof.

In some embodiments, the invention relates to a method of isolating an açai fruit oil comprising: a) providing a composition comprising açai mesocarp, b) mixing said composition with an extraction solution comprising a ketone and an alcohol such that a second solution comprising a set of insoluble components is formed; c) filtering said second solutions to separate said set of insoluble components providing a third solution; and d) isolating an açai fruit oil by removing volatile components from said third solution. In further embodiments, said extraction solution comprises between 10% to 50% acetone by volume and 50% to 90% ethanol by volume. In further embodiments, said extraction solution comprises between 30% to 50% acetone by volume and 50% to 70% ethanol by volume. In further embodiments, said extraction solution comprises about 57% ethanol, 3% water, and about 40% acetone by volume. In further embodiments, said açai oil contains a total polyphenolics concentration of at least 1, 5, 10, 100, 500, 1,000, 1,200, 1,400, 2,000, or 2,500 mg per liter of oil. In further embodiments, said açai oil contains a total phytosterol concentration of at least 1, 5, 10, 100, 500, 1,000, 1,200, 1,400, 2,000, or 2,500 mg per liter of oil. In further embodiments, said composition comprises açai fruit exocarp. In further embodiments, said açai fruit oil has a moisture content of less than 0.5%.

In some embodiments, the invention relates to a method of isolating an açai fruit oil comprising: a) providing a composition comprising açai mesocarp having a first viscosity; b) mixing said composition and a carbohydrase under conditions such that a first solution comprising a first set of insoluble components is formed with a second viscosity that is less that said first viscosity; c) separating said first set of insoluble components from said first solution; d) mixing said insoluble components with an extraction solution comprising a ketone and alcohol such that a second solution comprising a second set of insoluble components is formed; e) filtering said second solutions to separate said second set of insoluble components providing a third solution; and f) isolating an açai fruit oil by removing volatile components from said third solution. In further embodiments, said carbohydrase is a cellulase. In further embodiments, said extraction solution comprises between 10% to 50% acetone by volume and 50% to 90% ethanol by volume. In further embodiments, said extraction solution comprises between 30% to 50% acetone by volume and 50% to 70% ethanol by volume. In further embodiments, said extraction solution comprises about 57% ethanol, 3% water, and about 40% acetone. In further embodiments, said composition comprises açai fruit exocarp. In further embodiments, said açai fruit oil has a moisture content of less than 0.5%.

In other embodiments, the invention relates to a composition comprising a component comprising açai mesocarp and an extraction solution comprising a ketone and an alcohol. In further embodiments, extraction solution comprises between 10% to 50% acetone by volume and 50% to 90% ethanol by volume. In further embodiments, said extraction solution comprises between 30% to 50% acetone by volume and 50% to 70% ethanol by volume. In further embodiments, said extraction solution comprises about 57% ethanol, 3% water, and about 40% acetone.

In some embodiments, the invention relates to an açai oil extract comprising a total polyphenolics concentration of at least 1, 5, 10, 100, 500, 1,000, 1,200, 1,400, 2,000, or 2,500 mg per liter of oil.

In other embodiments, the invention relates to an açai oil extract comprising a total phytosterol concentration of at least 1, 5, 10, 100, 500, 1,000, 1,200, 1,400, 2,000, or 2,500 mg per liter of oil.

In some embodiments, the invention relates to an açai oil extract comprising a beta-sitosterol concentration of at least 1, 5, 10, 100, 500, 1,000, 1,200, 1,400, 2,000, or 2,500 mg per liter of oil.

In other embodiments, the invention relates to a method of isolating an açai fruit oil comprising: a) providing a composition comprising açai mesocarp, b) mixing said composition with a first extraction solution comprising alcohol such that a second solution comprising a first set of insoluble components is formed; c) separating said first set of insoluble components from said second solution; d) mixing said first set of insoluble components with a second extraction solution comprising ketone such that a third solution comprising a second set of insoluble components is formed; e) filtering said third solution to separate said set of insoluble components providing a fourth solution; and f) isolating an açai fruit oil by removing volatile components from said fourth solution. In further embodiments, said first extraction solution comprises between 99% to 60% ethanol by volume and 1% to 40% acetone by volume. In further embodiments, said second extraction solution comprises between 99% to 60% acetone by volume and 1% to 40% ethanol by volume. In further embodiments, said first extraction solution comprises between 99% to 60% acetone by volume and 1% to 40% ethanol by volume. In further embodiments, said second extraction solution comprises between 99% to 60% ethanol by volume and 1% to 40% acetone by volume. In further embodiments, said açai fruit oil enriched in phytosterols comprises a total phytosterol concentration of at least 1, 5, 10, 100, 500, 1,000, 1,200, 1,400, 2,000, or 2,500 mg per liter of oil. In further embodiments, said açai fruit oil enriched in phytosterols comprises a beta-sitosterol concentration of at least 1, 5, 10, 100, 500, 1,000, 1;200, 1,400, 2,000, or 2,500 mg per liter of oil.

In some embodiments, the invention relates to a method of isolating an açai fruit oil comprising: a) providing a composition comprising açai mesocarp, b) mixing said composition with an extraction solution comprising alcohol such that a second solution comprising a set of insoluble components is formed; c) filtering said second solutions to separate said set of insoluble components providing a third solution; and d) isolating an açai fruit oil by removing volatile components from said third solution. In further embodiments, said extraction solution comprises between 60% to 100% ethanol by volume. In further embodiments, said açai fruit oil comprises a total polyphenolics concentration of at least 1, 5, 10, 100, 500, 1,000, 1,200, 1,400, 2,000, or 2,500 mg per liter of oil.

In some embodiments, the invention relates to a method of isolating an açai fruit oil comprising: a) providing a composition comprising açai mesocarp, b) mixing said composition with an extraction solution comprising ketone such that a second solution comprising a set of insoluble components is formed; c) filtering said second solutions to separate said set of insoluble components providing a third solution; and d) isolating an açai fruit oil by removing volatile components from said third solution. In further embodiments, said extraction solution comprises between 60% to 100% acetone by volume. In further embodiments, said açai fruit oil comprises a total phytosterol concentration of at least 1, 5, 10, 100, 500, 1,000, 1,200, 1,400, 2,000, or 2,500 mg per liter of oil.

In some embodiments, the invention relates to a method for extracting an oil from açai fruit and açai fruit by-products, comprising: a) providing: i) at least one açai fruit, and ii) a solvent composition comprising ethanol and acetone; b) mixing said fruit and said solvent composition under conditions such that said oil is extracted from said fruit, and c) recovering said oil by removing said solvent via evaporative means. In further embodiments, said oil contains a total polyphenolics concentration of at least 500 mg per liter of oil. In still further embodiments, said oil contains a total phytosterol concentration of at least 10, 100, 300, 500, 1,000, 1,200, 1,400, 2,000, or 2,500 mg per liter of oil.

In some embodiments, the invention relates to a method of extracting oils from a moist matrix, preferably a fruit composition or portion thereof containing greater than 5%, 10%, 15%, 20%, or 25% of water by weight, such as fresh pulp and extracting components using methods disclosed herein.

In other embodiments, the invention relates to a method of extracting components from açai fruit by drying the fruit, preferably to contain less than 10% by weight of water, and even more preferably so that the fruit or portions thereof contain less than 5% but greater than 0.5% or 1.0% by weight of water, then extracting the components of the dried fruit into an extraction solution containing an alcohol and a ketone.

In some embodiments, the invention relates to an oil composition comprising: greater than 50% by weight unsaturated or polyunsaturated fatty acids; between 1% and 5%, or between 5% and 10% by weight, or between 10% and 20% by weight, or between 20% and 30% by weight, or greater than 10% by weight saturated fatty acids; and between 0.01% and 0.1% by weight, or between 0.1% and 0.5% by weight, or between 0.5% and 1.5% by weight, or between 1% and 5% by weight, or between 5% and 10% by weight, or greater than 10% by weight of polyphenolics and between 0.01% and 0.1% by weight, or between 0.1% and 0.5% by weight, or between 0.5% and 1.5% by weight, or between 1% and 5% by weight greater than 5% by weight of phytosterols. In further embodiments, said oil composition comprises greater than 15% by weight of polyphenolics. In further embodiments, said polyphenolics comprise: protocatechuic acid (3,4-dihydroxybenzoic acid); procyanidin C1 (Epicatechin-(4.beta.-->8)epicatechin-(4.beta.-->8)epicatechin; p-hydroxybenzoic acid; (+)-catechin; vanillic acid; (−)-epicatechin; proanthocyanidin A2 ((+)-Epicatechin-(4.beta.-8,2.beta.-O-7)-epicatechin); and ferulic acid. In further embodiments, said phytosterols comprises beta-sitosterol, stigmasterol, and campesterol. In further embodiments, the oil composition is obtained from an açai fruit.

In other embodiments, the invention relates to a cosmetic product comprising the compositions disclosed herein.

In other embodiments, the invention relates to a food product or nutritional supplement comprising the compositions disclosed herein.

In other embodiments, the invention relates to a pharmaceutical composition comprising the compositions disclosed herein.

In other embodiments, the invention relates to methods of cooking food using compositions disclosed herein.

In some embodiments, the invention relates to a method comprising administering to a mammal a composition comprising: greater than 50% by weight unsaturated or polyunsaturated fatty acids; between 1% and 5%, or between 5% and 10% by weight, or between 10% and 20% by weight, or between 20% and 30% by weight, or greater than 10% by weight saturated fatty acids; and between 0.01% and 0.1% by weight, or between 0.1% and 0.5% by weight, or between 0.5% and 1.5% by weight, or between 1% and 5% by weight, or between 5% and 10% by weight, or greater than 10% by weight of polyphenolics and between 0.01% and 0.1% by weight, or between 0.1% and 0.5% by weight, or between 0.5% and 1.5% by weight, or between 1% and 5% by weight greater than 5% by weight of phytosterols. In further embodiments, the components are obtained from an açai fruit. In further embodiments, the method further comprises the step of diluting the composition prior to orally administering said composition. In further embodiments, said administration is oral.

In other embodiments, the invention relates to a method comprising administering to a mammal a composition comprising an oil comprising: greater than 50% by weight unsaturated or polyunsaturated fatty acids; between 1% and 5%, or between 5% and 10% by weight, or between 10% and 20% by weight, or between 20% and 30% by weight, or greater than 10% by weight saturated fatty acids; and between 0.01% and 0.1% by weight, or between 0.1% and 0.5% by weight, or between 0.5% and 1.5% by weight, or between 1% and 5% by weight, or between 5% and 10% by weight, or greater than 10% by weight of polyphenolics and between 0.01% and 0.1% by weight, or between 0.1% and 0.5% by weight, or between 0.5% and 1.5% by weight, or between 1% and 5% by weight greater than 5% by weight of phytosterols. In further embodiments, the oil is obtained from an açai fruit. In further embodiments, the method comprises the step of diluting the composition prior to orally administering said composition.

In other embodiments, the invention relates to a method of managing, preventing or treating cancer comprising a) providing a subject at risk for, showing or having symptoms of, or diagnosed with cancer and a composition comprising an açai oil having component compositions as disclosed herein, or isolated by methods disclosed herein, b) administering said composition to said subject. In further embodiments, symptoms of said subject are reduced. In other embodiments, the invention relates to an oil having a chromatographic profile through a reverse-phase column when diluted in a mixture of methanol and water substantially similar to FIG. 4B.

In other embodiments, the invention relates to a method of managing, preventing or treating cancer comprising a) providing a subject at risk for, showing or having symptoms of, or diagnosed with cancer and a composition comprising an extract of an açai oil comprising polyphenolic components, and b) administering said composition to said subject. In other embodiments, the invention relates to a method of managing, preventing or treating cancer comprising a) providing a subject at risk for, showing or having symptoms of, or diagnosed with cancer and a composition comprising an extract of an açai oil comprising phenolic acids, and b) administering said composition to said subject.

### BRIEF DESCRIPTION OF THE FIGURES

FIG. 1 shows data on solvent extraction trials to maximize polyphenolic recovery from an açai product.

FIG. 2 shows data comparing phytochemical composition of laboratory batch run versus larger-scale pilot run (from FIG. 1) extracted with ethanol.

FIG. 3 shows data comparing the content of açai pulp, açai pulp oil, and the processed extracted oil from the by-product of Example 1.

FIG. 4A shows a chromatogram of açai oil physically removed from açai pulp. FIG. 4 B shows a chromatogram of ethanol-extracted açai oil from an açai fruit by-product described in Example 1.

FIG. 5 shows data of non-anthocyanin polyphenolics present in açai juice and the ethanol-extracted açai oil.

FIG. 6 show data on cell mortality observed in HL-60 cells following introduction of various açai polyphenolics at several doses.

FIG. 7 shows data on the activation of caspase-3 over a dose and time response in the presence of the glycosidic fraction comprised of non-anthocyanin polyphenolics.

FIG. 8 shows a HPLC chromatogram of polyphenolics in phytochemical-rich extracts from açai pulp (A) and açai oil (B). Peak assignments: 1: protocatechuic acid; 2: p-hydroxybenzoic acid; 3: (+)-catechin; 4: vanillic acid; 5: syringic acid; 6 and 7: procyanidin dimers; 8: ferulic acid; 9 and 10: procyanidin dimers; 11 through 14: procyanidin trimers.

FIG. 9A shows percent changes in total HT-29 cell numbers expressed as a ratio to control cells following treatment of cells with açai pulp or açai oil polyphenolic extracts adjusted to different concentrations (expressed as μg of gallic acid equivalents, GAE/mL) for 48 hours. Error bars represent the standard error of the mean (n=6).

FIG. 9B shows intracellular levels of reactive oxygen species in HT-29 cells following treatment with açai pulp or açai oil polyphenolic extracts adjusted to different concentrations (expressed in micrograms of gallic acid equivalents, GAE/mL). Error bars represent the standard error of the mean (n=6).

FIG. 10 shows a HPLC chromatogram of polyphenolics present in the basolateral compartment of Caco-2 cell monolayers following incubation with açai pulp (A) and açai oil (B) extracts for 2 hours. Peak assignments: 1: protocatechuic acid; 2: p-hydroxybenzoic acid; 3: vanillic acid; 4: syringic acid; 5: ferulic acid.

FIG. 11 shows a HPLC chromatogram of phenolics present in a typical E. oleracea oil extract. Peak assignments: 1: protocatechuic acid; 2: p-hydroxybenzoic acid; 3: (+)-catechin; 4: vanillic acid; 5: syringic acid; 6 and 7: procyanidin dimers; 8: ferulic acid; 9 and 10: procyanidin dimers; 11 through 14: procyanidin trimers.

FIG. 12 shows percent changes in total soluble phenolic contents during storage (30 and 40° C.) of E. oleracea oil extracts adjusted to three different initial phenolic contents (“high”, “intermediate”, and “low”). Error bars represent the standard error of the mean (n=3).

FIG. 13 shows percent changes in antioxidant capacity during storage (30 and 40° C.) of E. oleracea oil extracts adjusted to three different initial phenolic contents (“high”, “intermediate”, and “low”). Error bars represent the standard error of the mean (n=3).

FIG. 14 shows percent changes in total soluble phenolics and antioxidant capacity in 100% E. oleracea oil extracts following heating (150 or 170° C.). Error bars represent the standard error of the mean (n=3).

Table I shows the concentration and relative abundance of polyphenolics present in açai pulp and açai oil extracts. (a) Values represent non-anthocyanin polyphenolic concentrations equivalent to single-strength açai oil and correspond to a 300-fold concentrated açai pulp. (b) Values with different letters between columns represent a significant difference (paired samples t test, p<0.05).

Table II shows average transport rates of polyphenolics from açai pulp and açai oil extracts from the apical to the basolateral side of Caco-2 cell monolayers, as a function of total soluble phenolic contents. (a) Values with different letters within rows are significantly different (LSD test, p<0.05). (b) Total soluble phenolic contents (μg of GAE), which represent the absolute polyphenolic amount loaded into the apical side of cell monolayers. These amounts are equivalent to 3, 7.5, 15, 30, and 45 mL of açai pulp and to 10, 25, 50, 100, and 150 μL of açai oil, respectively.

Table III shows transport percentages of polyphenolics from açai pulp and açai oil extracts, from the apical to the basolateral side of Caco-2 cell monolayers following incubation (2 hours, 37° C.), as a function of total soluble phenolic contents. (a) Values with different letters within rows are significantly different (LSD test, p<0.05). (b) Total soluble phenolic contents (μg of GAE), which represent the absolute polyphenolic amount loaded into the apical side of cell monolayers. These amounts are equivalent to 3, 7.5, 15, 30, and 45 mL of açai pulp and to 10, 25, 50, 100, and 150 μL of açai oil, respectively.

Table IV shows HPLC-ESI-(−)-MS″ analyses of phenolics in E. oleracea oil extracts. (a) Ions in bold indicate the most intense product ion, on which further MS analyses were conducted.

Table V shows concentration (mg/L) and relative abundance (%) of nonanthocyanin phenolics present in E. oleracea clarified pulp and oil extracts. (a) Values with different superscript letters between columns represent a significant difference (paired samples t test, p<0.05).

Table VI shows major phenolics present in E. oleracea oil extracts (mg/L) adjusted to three different phenolic levels: “high”, “intermediate”, and “low”.

Table VII shows percent phenolic losses in E. oleracea oil extracts adjusted to different phenolic levels following storage (t=10 weeks) at 20, 30, and 40° C. (a) Values with different letters within rows are significantly different (LSD test, p<0.05).

### DETAILED DESCRIPTION OF INVENTION

The invention relates to methods used in extracting oil from plants, plant fruits, and/or nuts, preferably fruits from plants of the family Arecaceae, or Palmae, even more preferably açai fruit. In some embodiments, the invention relates to methods of extracting the oil using an extraction solution comprising a volatile alcohol and volatile ketone. In other embodiments, the invention relates to oil from açai fruit and açai fruit by-products that contain enriched concentrations of phytochemicals.

The fruits of the açai palm are edible. Usually, each palm tree produces from 3 to 4 bunches of fruit, each bunch having from 3-6 kg of fruit. The round-shaped fruits typically appear in green clusters when immature and ripen to a dark, purple colored fruit. A viscous juice may be prepared by macerating the edible pulp with water followed by passing through a small-mesh finished screen. The puree is typically pasteurized, placed into barrels, and frozen for export. Purees are typically thawed for use in various consumer products from frozen treats to beverages. In many cases, the oil content of the final product is not desired, prompting its removal by various methods including physical removal, pressing from the pulp, cold-separation; filtration, or other methods to physically or chemically remove the oil. The resultant oil is usually dark-green in color but may contain extraneous contaminants from the açai pulp itself or contain water in a free or bound state.

As used herein, “oil” or “oil composition” refers to a liquid or gel-like composition that contains less than 1% by weight of water. “Arai fruit oil” refers to an oil containing components isolated from the fruit of açai palm. This oil may or may not contain residual water and solvent components used in extraction methods disclosed herein for obtaining the oil. It is not intended that açai fruit be limited to the berry of a naturally occurring plant and may be derived from a plant genetically modified through genetic engineering or cross breeding. Typically, the fruit has a single large seed about 7-10 mm in diameter. The exocarp, or skin, of the ripe açai fruits is usually a deep purple color, or green, depending on the kind of açai and its maturity. The mesocarp, or pulp, surrounds the voluminous and hard endocarp which contains a seed with a embryo and endosperm. A “composition comprising açai mesocarp” refers to any composition that contains the açai mesocarp, and it is not intended to be limited to pulpy composition obtained directly from the berry. It is intended to include composition obtained after manipulating the pulp, e.g., when the pulp is dried and milled, including those compositions that contain insoluble constituents that remain after extraction. It is not intended to be limited to composition that contains only pulp but may also contain, for example, the skin or seed of the açai fruit or residual components that occur after manipulating the pulp.

As used herein an “isolated non-naturally occurring” component(s), such as an oil, means that the component(s) have been collected by human physical intervention. It is contemplated that the components may be isolated from a plant or other natural product which may be obtained from a cultivated or uncultivated areas, i.e., the terms are intended to include components isolated from natural sources, but it is not intended to include the compositions that exist in the plants themselves without isolation.

In some embodiments, the invention relates to an açai oil product derived from any embodiment of the açai berry, extract of the açai berry, or by-product of the açai berry or açai berry processing operations. By-products include those from fruit pulping, clarification, extraction, physical separation, centrifugation, etc., inclusive of the remaining açai seed following fruit pulping and any residual oil still clinging to the seed. Organic solvents, organic alcohols, ketones, or non-solvent techniques are used to extract and isolate oil from the açai berry or açai berry processing operations. Solvent processes include petroleum distillates such as hexane, isohexane, or any other hydrocarbon solvent suitable for extracting açai oil. Organic alcohols include ethanol, methanol, propanol, isopropanol, or any other organic alcohol suitable for extracting açai oil. Ketones primarily refer to acetone or similar solvents for extracting açai oils. Non-solvent techniques refer to physical removal of açai oil, pressing of açai seeds, pressing of dehydrated açai, or the use of supercritical gases such as carbon dioxide for açai oil extraction. These methods, especially in the presence of açai fruit, açai pulp, or açai by-products allow for extraction of açai oil or açai oil containing other phytochemical constituents such as phytosterols, polyphenolics, tocopherols, carotenoids, and other phytochemical compounds. All traces of solvent are removed in the process, leaving behind an oil that contains no appreciable traces of the solvent and that contains <0.5% water by weight.

The resultant oil is dark green in color and consists of approximately 60-80% monounsaturated fatty acids, 10-20% polyunsaturated fatty acids, and 10-20% saturated fatty acids. The phytochemical content and composition of this oil is unique to other oils. The derived oil can be further processed, refined, or deodorized to remove part of all of the color and/or phytochemical constituents and can be refined for food, drug, dietary supplement, industrial, or cosmetic grade products.

Applications of fruit oil include the preparation of water-soluble oils using emulsifiers that can then be incorporated into food and cosmetic products. These oils may be combined with compounds including but not limited to carotenoids, fat-soluble vitamins, essential oils, plant extracts and other bioactive agents. Furthermore, the oil may be treated to form a solid or treated in such a way to keep the oil a liquid when refrigerate in a processes known as winterization. The oil may also be refined as needed for use in commercial products. Finally, the oil may be combined with any other food or cosmetic grade oil as a dilution agent, including but not limited to grape seed oil, pomegranate seed oil, raspberry seed oil, olive oil and sunflower seed oil, for processing to suit food, dietary supplement and cosmetic applications.

International commercialization of açai fruit imposes new challenges since various processing steps such as pasteurization, freezing, dilution, fortification and/or dehydration may be used during manufacture of retail products. U.S. Patent Publication No. 2006/0275511, hereby incorporated by reference, provides for a number of uses of açai fruit compositions. Many juices are fortified with ascorbic acid to slow browning reactions as well as to enhance nutritional properties. In certain embodiments, the invention relates to oil recovered froth açai fruit and fruit by-products. Several processes for extracting anthocyanosides from plants or from plant portions have been proposed and most utilize mixtures of ethanol and water. See e.g., Gallori et al., Chromatographia, 2004, 59(11/12), 739-743. However, the Applicants have not identified a disclosure that focuses on maximizing açai oil extraction for açai oil compositions.

U.S. Pat. No. 6,461,648 discloses a process for the purification of red fruit extract containing anthocyanosides by obtaining a pre-purified extract taken up in methanol, filtered through an absorbent macrocrosslinked resin and eluted from the resin with an aqueous solution of ethanol whose ethanol concentration is between 10% and 90%.

Document FR-A-2 299 385 describes a process for extracting anthocyanidins from grape marc comprising an extraction step proper, followed by a step for concentrating the extract obtained. According to this process, the extraction step consists in treating the marcs with an acidic aqueous extraction solution (pH=2) supplemented with SO₂ in the hot state (between 40 and 55° C.). The clear solution obtained, containing the anthocyanosides, but also the acids, salts, polyphenols and proteins, is then concentrated. To do this, it is loaded onto a resin. The resin is then eluted with an eluting solution containing either a ketone, an amide or an aqueous solution of an alkali or alkaline-earth metal hydroxide. The anthocyanins are finally separated from the eluate obtained. The initial extraction of the anthocyanosides with a solvent supplemented with SO₂ leads to the attachment of the anthocyanosides to the resin in a modified form, which is therefore capable of disrupting the physicochemical characteristics of the anthocyanoside and therefore its activity.

A process for extracting and then purifying anthocyanosides from bilberries was proposed in the document EP-A-412 300. The extraction step consists of placing frozen fruits in contact with an aqueous solution of methanol, each extraction extending over a period of four hours. The extract obtained is then purified. To do this, it is first concentrated under vacuum, the resulting concentrate then being supplemented with sodium bisulfite. A bond is then formed between the anthocyanosides and the bisulfite ions. After stirring for three hours and neutralization by adding a sodium hydroxide solution, the extract obtained is loaded onto a column of a polymer resin and then the column is eluted with purified water. The eluate is then acidified to pH=1 with concentrated hydrochloric acid (HCl). To remove the SO₂, nitrogen is then bubbled through the solution obtained so as to dissociate the anthocyanosides-bisulfite complex. This dissociation leads to the release of sulfur dioxide. The aqueous solution is then extracted with butanol. The butanolic solution is supplemented with 14 volumes of ethyl acetate. After allowing the solution to stand overnight, the precipitate is dried at 40° C.

The Applicants have identified an improved method of extracting açai oils for the purpose of solubilizing phytochemicals while simultaneously maximizing oil recovery, e.g., polyphenolics, phytosterols, lipids, fatty acids, and porphyrin, using mixtures of a volatile alcohol and a volatile ketone. Processes were developed primarily using ethanol and acetone as the extracting solvents to capture not only the lipids from the açai fruit, but also many of the phytochemical components of the açai fruit, many of which have antioxidative properties. The resulting açai oil may contain anthocyanins, phenolic acids, flavonoids, tocopherols, carotenoids, phytosterols, and other chemical constituents. The oil is also high in the mono-unsaturated fatty acid oleic acid (C18:1; omega-9 fatty acid) and contains other polyunsaturated fatty acids such as linoleic acid and gamma-linolenic acid (C18:2 and C18:3; omega-6 fatty acids). The high polyphenolic content of the oil varies based on the extraction method and is higher than the açai fruit from which it was derived due to processing enhancements. It is also possible to isolate an oil with substantially no polyphenolics. Many of these polyphenolic compounds are antioxidant agents and compounds such as anthocyanins (cyanidin-3-rutinoside and cyanidin-3-glucoside), phenolic acids (gallic acid and gallotannins, protocatechuic acid, p-hydroxybenzoic acid, vanillic acid, and ferulic acid), and flavonoids ((+)-catechin, (−)-epicatechin, and various condensed tannins) have been identified in the fruit or in oil extract.

Depending on the oil extraction and procesing techniques, the polyphenolics present are expected to vary. Among the antioxidant polyphenolics present in açai fruit, the anthocyanins generally contribute the greatest impact to total antioxidant capacity. The remaining phenolic acids and flavonoids (also referred to as non-anthocyanin polyphenolics) were previously reported to account for as little as 5% to as much as 33% for the total antioxidant capacity of the açai fruit. Although anthocyanins may be present in the oil, the described extraction processes are exceptionally efficient at extracting and enhancing concentrations of non-anthocyanin polyphenolics in the oil. The presence of non-anthocyanin polyphenolics were also shown to enhance the color of anthocyanins in solution by ˜6%, exert a protective effect against ascorbic acid-induced degradation of anthocyanins (Pacheco-Palencia et al. 2007), and were also shown to enhance the half-life of anthocyanins color in solution from 319 to 385 hours when held at 37° C. During açai juice or açai pulp shelf-life evaluations, the non-anthocyanin polyphenolics were found to be very stable overall, with generally <5% degradation over 12 days at 37° C.

As used herein, “administering” refers to apply to the skin or orally feeding yourself or someone else an identified composition.

As used herein, a “ketone” refers to any variety of compounds that contain a carbonyl group disubstituted with alkyl groups, i.e., RC=OR. It is not intended that both alkyl groups must be the same alkyl. In preferred embodiments of the invention, extractions are performed with the ketone, acetone, wherein the alkyl groups are methyl groups. Other possible alkyl groups include ethyl, propyl, butyl, isopropyl and isobutyl groups.

As used herein, an “alcohol” solvent refers to any variety of compounds that contain an alkyl group substituted with a hydroxyl group, i.e., ROH. In preferred embodiments of the invention, extractions are performed with the alcohol, ethanol, wherein the alkyl groups are ethyl groups. It is contemplated that the ethanol solvent contains limited amounts of water, and it is not intended that the term be limited to anhydrous ethanol unless otherwise specified as anhydrous ethanol.

As used herein, a “solvent” refers to any liquid composition in relation to an identified composition to be partially or totally dissolved in the liquid. For example, water is a solvent for a variety of substances such as salts and monasaccharide. In reference to a solvent containing 100% of a specific liquid, it is contemplated that this solvent may contain small amounts of residual impurities or water that is absorbed from the atmosphere preferably of less than 0.5% and even more preferably of less than 0.1%.

As used herein, “volatile” solvents, components, or compounds refers to any of a variety of chemical compounds that have high enough vapor pressures at about 10 mm Hg (wherein 760 mm is atmspheric pressure) at room temperature to eventually turn into a gas.

“Polyphenolics” refers to any of a variety of compounds that have an aromatic group substituted with one or more hydroxyl groups. Preferred polyphenolics include, for example, flavonoids, anthocyanidins, proanthocyanidins, and free and esterified phenolic acids such as gallic acid.

As used herein, a “phytosterol” refers to any derivative or substituted compounds having the A, B, C, and D rings of the steroid skeleton. Preferred phytosterols include beta-sitosterol, stigmasterol, campesterol, and ergosterol.

As used herein, “viscosity” refers to a measure of the resistance of a fluid to deform under shear stress.

“Carbohydrase” refers to an enzyme that hydrolyzes polymers made up of various types of five- or six-carbon sugars as the backbone of the polymer. Examples include cellulases that produce glucose from cellulose. Another example is lactase, which hydrolyses lactose to glucose and galactose.

The term “manage” when used in connection with a disease or condition means to provide beneficial effects to a patient being administered with a prophylactic or pharmaceutical composition, which does not result in a cure of the disease. In certain embodiments, a patient is administered with one or more prophylactics or compositions to manage a disease so as to prevent the progression or worsening of the disease.

As used herein, the terms “prevent” and “preventing” include the prevention of the recurrence, spread or onset. It is not intended that the present invention be limited to complete prevention. In some embodiments, the onset is delayed, or the severity of the disease is reduced.

“Subject” means any animal, preferably a human patient, livestock, or domestic pet.

As used herein, the terms “treat” and “treating” are not limited to the case where the subject (e.g. patient) is cured and the disease is eradicated. Rather, the present invention also contemplates treatment that merely reduces symptoms, and/or delays disease progression.

### Pharmaceutical Compositions

The compositions comprising oils disclosed include bulk-drug compositions useful in the manufacture of pharmaceutical compositions (e.g., impure or non-sterile compositions) and pharmaceutical compositions (i.e., compositions that are suitable for administration to a patient) that can be used in the preparation of unit dosage forms. Such compositions optionally comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of the active compound and another therapeutic or prophylactic agent, and a pharmaceutically acceptable carrier.

In a specific embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the oil is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a patient, the pharmaceutically acceptable vehicles are preferably sterile. Water can be the vehicle when the oil is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

The present compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155), such as a gel capsule.

In a preferred embodiment, the oil and optionally another therapeutic or prophylactic agent are formulated in accordance with routine procedures. Typically, the oils for intravenous administration are solutions in sterile isotonic aqueous buffer. Where necessary, the compositions can also include a solubilizing agent. Compositions for intravenous administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the oil is to be administered by infusion, it can be dispensed, for example, with an infusion bottle containing sterile pharmaceutical grade water or saline. Where the oil is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract, thereby providing a sustained action over an extended period of time. Selectively permeable membranes surrounding an osmotically active driving compound are also suitable for an oral administration of the oil. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like. Such vehicles are preferably of pharmaceutical grade.

Further, the effect of the oil can be delayed or prolonged by proper formulation. For example, a slowly soluble pellet of the oil can be prepared and incorporated in a tablet or capsule. The technique can be improved by making pellets of several different dissolution rates and filling capsules with a mixture of the pellets. Tablets or capsules can be coated with a film that resists dissolution for a predictable period of time. Even the parenteral preparations can be made long acting, by dissolving or suspending the compound in oily or emulsified vehicles, which allow it to disperse only slowly in the serum.

Pharmaceutical compositions for use in accordance with the present invention can be formulated in a conventional manner using one or more physiologically acceptable carriers or excipients.

Thus, the compound and optionally another therapeutic or prophylactic agent and their physiologically acceptable salts and solvates can be formulated into pharmaceutical compositions for administration by inhalation or insufflation (either through the mouth or the nose) or oral, parenteral or mucosol (such as buccal, vaginal, rectal, sublingual) administration. In one embodiment, local or systemic parenteral administration is used.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup; cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the oil.

For buccal administration the pharmaceutical compositions can take the form of tablets or lozenges formulated in conventional manner.

For administration by inhalation, the pharmaceutical compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical compositions can be formulated for parenteral administration by injection, e.g., by bolus injection or continuous infusion. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The pharmaceutical compositions can also be formulated in rectal compositions such as suppositories or retention enemas, e.g., containing conventional suppository bases such as cocoa butter or other glycerides.

In addition to the formulations described previously, the pharmaceutical compositions can also be formulated as a depot preparation. Such long acting formulations can be administered by implantation (for example subcutaneously or intramuscularly) or by intramuscular injection. Thus, for example, the pharmaceutical compositions can be formulated with suitable polymeric or hydrophobic materials (for example, as an emulsion in an acceptable oil) or ion exchange resins, or as sparingly soluble derivatives, for example, as a sparingly soluble salt.

The invention also provides that a pharmaceutical composition is packaged in a hermetically sealed container such as an ampoule or sachette indicating the quantity. In one embodiment, the pharmaceutical composition is supplied as a dry sterilized lyophilized powder or water free concentrate in a hermetically sealed container and can be reconstituted, e.g., with water or saline to the appropriate concentration for administration to a patient.

In other embodiments of the invention, radiation therapy agents such as radioactive isotopes can be given orally as liquids in capsules or as a drink. Radioactive isotopes can also be formulated for intravenous injection. The skilled oncologist can determine the preferred formulation and route of administration.

The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

In certain preferred embodiments, the pack or dispenser contains one or more unit dosage forms containing no more than the recommended dosage formulation as determined in the Physician's Desk Reference (56^{th} ed. 2002, herein incorporated by reference in its entirety).

Methods of administering the oil and optionally another therapeutic or prophylactic agent include, but are not limited to, parenteral administration (e.g., intradermal, intramuscular, intraperitoneal, intravenous and subcutaneous), epidural, and mucosal (e.g., intranasal, rectal, vaginal, sublingual, buccal or oral routes). In a specific embodiment, the oil and optionally other prophylactic or therapeutic agents are administered intramuscularly, intravenously, or subcutaneously. The oil and optionally another prophylactic or therapeutic agent can also be administered by infusion or bolus injection and can be administered together with other biologically active agents. Administration can be local or systemic. The oil and optionally the prophylactic or therapeutic agent and their physiologically acceptable salts and solvates can also be administered by inhalation or insufflation (either through the mouth or the nose). In a preferred embodiment, local or systemic parenteral administration is used.

In specific embodiments, it can be desirable to administer the oil locally to the area in need of treatment. This can be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers. In one embodiment, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In certain embodiments, the oil can be formulated as a suppository, with traditional binders and vehicles such as triglycerides.

In another embodiment, the oil can be delivered in a vesicle, in particular a liposome.

In yet another embodiment, the oil can be delivered in a controlled release system. In one embodiment, a pump can be used. In another embodiment, polymeric materials can be used.

The amount of the oil that is effective in the treatment or prevention of heart conditions can be determined by standard research techniques. For example, the dosage of the oil that will be effective in the treatment or prevention of heart conditions can be determined by administering the oil to an animal in a model such as, e.g., the animal models known to those skilled in the art. In addition, in vitro assays can optionally be employed to help identify optimal dosage ranges.

Selection of a particular effective dose can be determined (e.g., via clinical trials) by a skilled artisan based upon the consideration of several factors, which will be known to one skilled in the art. Such factors include the disease to be treated or prevented, the symptoms involved, the patient's body mass, the patient's immune status and other factors known by the skilled artisan.

The precise dose to be employed in the formulation will also depend on the route of administration, and the seriousness of the disease-related wasting, and should be decided according to the judgment of the practitioner and each patient's circumstances. Effective doses can be extrapolated from dose-response curves derived from in vitro or animal model test systems.

The dose of the oil to be administered to a patient, such as a human, is rather widely variable and can be subject to independent judgment. It is often practical to administer the daily dose of the oil at various hours of the day. However, in any given case, the amount of the oil administered will depend on such factors as the solubility of the active component, the formulation used, patient condition (such as weight), and/or the route of administration.

The general range of effective amounts of the oil alone or in combination with another prophylactic or therapeutic agent(s) are from about 0.001 mg/day to about 1000 mg/day, more preferably from about 0.001 mg/day to 750 mg/day, more preferably from about 0.001 mg/day to 500 mg/day, more preferably from about 0.001 mg/day to 250 mg/day, more preferably from about 0.001 mg/day to 100 mg/day, more preferably from about 0.001 mg/day to 75 mg/day, more preferably from about 0.001 mg/day to 50 mg/day, more preferably from about 0.001 mg/day to 25 mg/day, more preferably from about 0.001 mg/day to 10 mg/day, more preferably from about 0.001 mg/day to 1 mg/day. Of course, it is often practical to administer the daily dose of compound in portions, at various hours of the day. However, in any given case, the amount of compound administered will depend on such factors as the solubility of the active component, the formulation used, subject condition (such as weight), and/or the route of administration.

In some embodiments, the invention provides a pharmaceutical pack or kit comprising one or more containers containing an oil and optionally one or more other prophylactic or therapeutic agents useful for the prevention or treatment of cancer. The invention also provides a pharmaceutical pack or kit comprising one or more containers containing one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration; or instructions for the composition's use.

### Cosmetic Compositions

The cosmetic and dermatological preparations according to the invention can comprise cosmetic active ingredients, auxiliaries and/or additives, for example, coemulsifiers, fats and waxes, stabilizers, thickeners, biogenic active ingredients, film formers, fragrances, dyes, pearlizing agents, preservatives, pigments, electrolytes (e.g. magnesium sulfate) and pH regulators.

Suitable coemulsifiers are, for example, polyglycerol esters, sorbitan esters or partially esterified glycerides. Typical examples of fats are glycerides; waxes that may be mentioned are inter alia beeswax, paraffin wax or microcrystalline waxes, optionally in combination with hydrophilic waxes. Stabilizers that may be used are metal salts of fatty acids, such as, for example, magnesium, aluminum and/or zinc stearate. Examples of suitable thickeners are crosslinked polyacrylic acids and derivatives thereof, polysaccharides, in particular xanthan gum, guar gum, agar agar, alginates and tyloses, carboxymethylcellulose and hydroxyethylcellulose, and also fatty alcohols, monoglycerides and fatty acids, polyacrylates, polyvinyl alcohol and polyvinylpyrrolidone. The term biogenic active ingredients means, for example, plant extracts, protein hydrolyzates and vitamin complexes. Customary film formers are, for example, hydrocolloids, such as chitosan, microcrystalline chitosan or quaternary chitosan, polyvinylpyrrolidone, vinylpyrrolidone/vinyl acetate copolymers, polymers of the acrylic acid series, quaternary cellulose derivatives and similar compounds. Examples of suitable preservatives are formaldehyde solution, p-hydroxybenzoate or sorbic acid. Examples of suitable pearlizing agents are glycol distearic esters, such as ethylene glycol distearate, but also fatty acids and fatty acid monoglycol esters. Dyes which may be used are the substances suitable and approved for cosmetic purposes. These dyes are usually used in a concentration of from 0.001 to 0.1% by weight, based on the total mixture.

It is likewise advantageous to add customary antioxidants to the preparations for the purposes of the present invention. According to the invention, all antioxidants that are customary or suitable for cosmetic and dermatological applications may be used as favorable antioxidants.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, carnosine, L-carnosine and derivatives thereof (e.g. anserine), carotenoids, carotenes (e.g. alpha-carotene, beta-carotene, lycopene) and derivatives thereof, retinoids, such as, for example, retinol, retinal and/or retinoic acid and the respective esters, alpha-lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, gamma-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. alpha-hydroxy fatty acids, palmitic acid, phytic acid, lactoferrin), alpha-hydroxy acids (e.g. citric acid, lactic acid, maleic acid), humic acid, bile acid, bile extracts, bilirubin, biliverdin, EDTA, EGTA and derivatives thereof, unsaturated fatty acids and derivatives thereof (e.g., gamma-linolenic acid, linoleic acid, oleic acid), folic acid and derivatives: thereof, 2-aminopropionic acid, diacetic acid, flavonoids, polyphenols, catechins, ubiquinone and ubiquinol and derivatives thereof, vitamin C and derivatives (e.g. ascorbyl palmitate, magnesium ascorbyl phosphate, ascorbyl acetate), tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaiacic acid, nordihyrdoguaiaretic acid, trihydroxybutyrophenone, uric acid and derivatives thereof, mannose and derivatives thereof, zinc and derivatives thereof, (e.g. ZnO, ZnSO₄), selenium and derivatives thereof (e.g. selenomethionine), stilbene and derivatives thereof (e.g. stilbene oxide, trans-stilbene oxide) and the derivatives (salts, esters, ethers, sugars, nucleotides, nucleosides, peptides and lipids) of said active ingredients which are suitable according to embodiments of the invention.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or antioxidants, it is advantageous to choose their respective concentrations from the range 0.001 to 10% by weight, based on the total weight of the formulation.

If vitamin A and/or derivatives thereof or carotenoids are the antioxidant or antioxidants, it is advantageous to choose the respective concentration thereof from the range 0.001 to 10% by weight, based on the total weight of the formulation.

If the cosmetic or dermatological preparation is a solution or emulsion or dispersion, the solvents used may be: water or aqueous solutions; oils, such as triglycerides of capric acid or of caprylic acid, but preferably castor oil; fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids; alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the above mentioned solvents are used. In the case of alcoholic solvents, water may be a further constituent.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, diisopropyl adipate, n-hexyl laurate, n-decyl oleate, glyceryl stearate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semi-synthetic and natural mixtures of said esters, e.g. jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids with a chain length of from 8 to 24 carbon atoms, in particular 12 to 18 carbon atoms. The fatty acid triglycerides can, for example, be chosen advantageously from the group of synthetic, semisynthetic and natural oils, e.g. olive oil, sunflower oil, soybean oil, peanut oil, rapeseed oil, almond oil, palm oil, coconut oil, palm kernel oil and the like.

Any mixtures of such oil and wax components are also to be used advantageously. It may in some instances also be advantageous to use waxes, for example cetyl palmitate, as the sole lipid component of the oil phase.

The oil phase is advantageously chosen from the group consisting of 2-ethylhexyl isostearate, isohexadecane, octyldodecanol, isotridecyl isononanoate, isoeicosane, 2-ethylhexyl cocoate, C₁₂-C₁₅-alkyl benzoate, caprylic/capric acid triglyceride, dicaprylyl ether.

Mixtures of C₁₂-C₁₅-alkyl benzoate and 2-ethylhexyl isostearate, mixtures of C₁₂-C₁₅-alkyl benzoate and isotridecyl isononanoate, and mixtures of C₁₂-C₁₅-alkyl benzoate, 2-ethylhexyl isostearate and isotridecyl isononanoate are particularly advantageous.

Of the hydrocarbons, paraffin oils, squalane and squalene are to be used advantageously for the purposes of the present invention.

Advantageous oil components are also, for example, butyloctyl salicylate (for example that available under the trade name Hallbrite BHB from CP Hall), hexadecyl benzoate and butyloctyl benzoate and mixtures thereof (Hallstar AB) and/or diethylhexyl naphthalate (Hallbrite TQ).

The oil phase can also advantageously have a content of cyclic or linear silicone oils, or consist entirely of such oils, although it is preferred to use an additional content of other oil phase components apart from the silicone oil or the silicone oils.

Advantageously, cyclomethicone (octamethylcyclotetrasiloxane) is used as silicone oil to be used according to the invention. However, other silicone oils can also be used advantageously for the purposes of the present invention, for example hexamethylcyclotrisiloxane, polydimethylsiloxane, poly(methylphenylsiloxane).

Mixtures of cyclomethicone and isotridecyl isononanoate, and of cyclomethicone and 2-ethylhexyl isostearate are also particularly advantageous.

Solid sticks comprise, for example, natural or synthetic waxes, fatty alcohols or fatty acid esters. Preference is given to using lip care sticks, and stick formulations for deodorizing the body.

Customary basic substances that are suitable for use as cosmetic sticks for the purposes of the present invention are liquid oils (e.g. paraffin oils, castor oil, isopropyl myristate), semisolid constituents (e.g. petroleum jelly, lanolin), solid constituents (e.g. beeswax, ceresin and microcrystalline waxes and ozocerite), and high-melting waxes (e.g. carnauba wax, candelilla wax).

Suitable propellants for cosmetic and/or dermatological preparations for the purposes of the present invention that can be sprayed from aerosol containers are the customary known, readily volatile, liquefied propellants, for example hydrocarbons (propane, butane, isobutane), which can be used on their own or in a mixture with one another. Compressed air can also be used advantageously.

The person skilled in the art is of course aware that there are propellant gases which are nontoxic per se which would in principle be suitable for realizing the present invention in the form of aerosol preparations, but which nevertheless have to be avoided due to a harmful effect on the environment or other accompanying phenomena, in particular fluorocarbons and chlorofluorocarbons (CFCs).

Cosmetic preparations for the purposes of the present invention may also be in the form of gels which, besides an effective content of active ingredient according to the invention and solvents customarily used therefore, preferably water, also comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxpropylmethylcellulose or inorganic thickeners, e.g. aluminum silicates, such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight. The cosmetic and pharmaceutical preparations comprising light protection agents are generally based on a carrier that comprises at least one oil phase. Preparations based solely on aqueous components are, however, also possible. Accordingly, suitable preparations are oils, oil-in-water and water-in-oil emulsions, creams and pastes, lip-protection stick compositions or grease-free gels.

Gels used according to the invention usually comprise alcohols of low carbon number, e.g. ethanol, isopropanol, 1,2-propanediol, glycerol and water or an above mentioned oil in the presence of a thickener, which in the case of oily-alcoholic gels is preferably silicon dioxide or an aluminum silicate, and in the case of aqueous-alcoholic or alcoholic gels is preferably a polyacrylate.

The total proportion of auxiliaries and additives can be 1% to 80% by weight, preferably 6% to 40% by weight, and the nonaqueous proportion (“active substance”) can be 20% to 80% by weight, preferably 30% to 70% by weight, based on the compositions. The compositions can be prepared in a manner known per se, i.e. for example by hot, cold, hot-hot/cold or PIT emulsification. This is a purely mechanical process, and no chemical reaction takes place.

### EXAMPLES

The following examples are illustrative embodiments and are in no way intended to limit the scope of embodiments of the invention.

### Example 1

One açai clarification process involves receiving frozen açai pulp in plastic-lined 55-gallon drums and thawing the pulp at 10° C. (55° F.) until a 12×24 inch block of ice remains in the drum. Drums are then individually dumped into a holding tank whereby the temperature is brought to 43° C. (110° F.), and may range from 25-65° C. Cellular degrading enzymes are then added at a rate of 9 liters of Validase TRL cellulose enzyme (Valley Research, South Bend, Ind.) per 1,000 gallons of açai pulp and blended. The enzyme system may be any equivalent or mixture of cellulase, pectinase, or hemicellulase since most enzyme preparations used in food applications are not pure. Enzyme use can range from 2-20 liters per 1,000 gallons of açai pulp. The enzyme is mixed thoroughly with the pulp and allowed to incubate for 4 hours, where incubation times can range from 0.5-12 hours. Following incubation, the pulp is then rapidly heated to 71° C. (160° F.) to denature the enzyme. The denaturation can range in temperature from 60-80° C. The pulp is then rapidly chilled to 10° C. (50° F.) or colder to facilitate the removal of lipids. The pulp is then partially clarified on a rotary vacuum filter system through a layer of diatomaceous earth (FW 20 or equivalent). This process removes a majority of insoluble solids and filterable lipids.

The semi-clarified juice is then chilled to 1.6° C. (35° F.), ranging from 1-8° C., and held in a settling tank for 48-72 hours to allow for solidification of remaining lipids. Time in the settling tank can vary from 2 hours to 5 days. The settling tank is then drained into a pressure leaf filter containing a smaller size diatomaceous earth (FW 12 or equivalent) where lipids are reduced to 0.05% or less and an NTU value (Nephelometric Turbidity Units) of <50. The range, of lipids can vary from 0% to 0.25% in the finished product and an NTU value from 50-1,000. A final filtration through a 20-μm filter sock is conducted immediately prior to transportation to the bottling facility. Typical processes recover from 60% to 100% juice form the initial pulp by weight or based on the recovery of anthocyanin-based color.

### Example II

Tests for total soluble phenolics involved the use of the Folin-Ciocalteu assay for total soluble phenolics, whereby the total reducing capacity of isolates were compared against a standard of gallic acid by spectrophotometric assay. Measurement of total hydrophilic antioxidant capacity in pulp or oil was done following dilution of the açai pulp/juice and/or extraction of the antioxidants from the oil with a mixture of 50% methanol and 50% water. After appropriate dilutions, the ability of the plant extracts to inhibit the decay of fluorescein in the presence of a peroxyl radical over 70 minutes time at 37° C. was measured against a standard curve of Trolox, a water-soluble analog of Vitamin E, using a microplate reader. Data are presented at μmol Trolox equivalents per mL (or per kilogram) of pulp/juice/oil. HPLC analysis of individual polyphenolics was conducted on a Waters 2695 system and detected at 280 nm with a photodiode array detector. Compounds were quantified using external standards of the respective compounds.

The açai oil process and composition described herein may be extracted with a blend of polar organic solvents to maximally recover both phytochemicals and the oil itself from açai fruit products or açai fruit by-products. Average oil yields may range from 2% to 20% depending on the source for extraction. Traditional oil extraction technologies such as cold pressing, physical recovery of the oil, or non-polar solvents will not recover significant quantities of the polyphenolics present in the source material (açai pulp or process by-product) or from the naturally-occurring oil present in the açai fruit. The composition of açai oil beyond its fatty acid content is important for uses in products such as dietary antioxidants for food, supplement, drug, and cosmetic applications. In addition to the benefits of the polyphenolics, its high phytosterol and mono/polyunsaturated fatty acids (omega 6 and 9 fatty acids) have bee associated with decreased risk for coronary heart disease and other chronic diseases. The oil contains very little to no trans-fats.

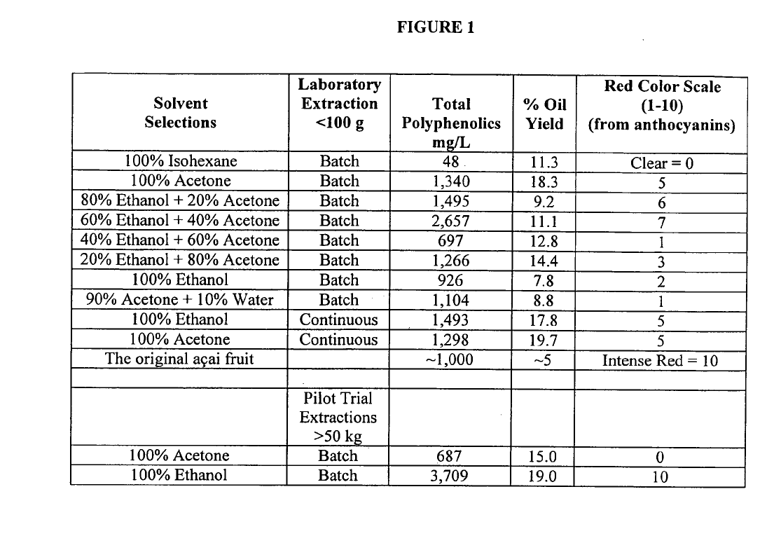

Various mixtures of food-grade organic solvents (95% ethanol and 100% acetone) were investigated on a laboratory scale. The insoluble solids and filterable lipids of Example 1 where transferred to extraction solutions of isohexane, acetone, ethanol (absolute ethanol (190 proof) unless otherwise indicated) and acetone in a 4:1 mix by volume, ethanol and acetone in a 3:2 mix by volume, ethanol and acetone in a 1:4 mix by volume, ethanol and acetone in a 2:3 mix by volume, ethanol, acetone and water in a 10:1 mix by volume. After about one hour of soaking the solutions, residual solids are filtered, and volatile solvents are removed under vacuum. Residual moisture was removed by centrifugation. A variation was done by soxhlet extraction using ethanol and acetone. Total polyphenolics concentrations from the extractions are provided in FIG. 1. Pure solvents and solvent mixtures were investigated in a batch and continuous extraction system and evaluated for total soluble polyphenolics, oil recovery, and a subjective score of anthocyanin recovery based on red color intensity.

By using extraction solutions using an alcohol and/or a ketone one obtains oils comprising approximately 60-80% monounsaturated fatty acids, 10-20% polyunsaturated fatty acids, and 10-20% saturated fatty acids, which traps and solubilizes the phytochemicals that were present in the fruit. The phytochemical content of the oil is unique since most oil extractions do not recover antioxidant phytochemicals such as polyphenolics since it is often difficult to remove water from the polyphenolics that are generally hydrophilic. Polyphenolics identified include protocatechuic acid, procyanidin, p-hydroxybenzoic acid, (+)-catechin, vanillic acid, (−)-epicatechin, procyanidin-2, and ferulic acid.

Oil recovery rates were in inverse correlation with the polyphenolics, i.e., the more ethanol that was used recovered higher polyphenolics concentrations but less oil and the more acetone that was used recovered more oil and fewer polyphenolics. The solution of 3:2 ratio of ethanol to acetone seemed to be optimal for recovering a large amount of polyphenolics and oil. In certain embodiments, it is contemplated that one can extract the oil with a high concentration of ethanol to obtain an oil laden with polyphenolics for dietary supplements, cosmetics, and other high-end products. The insoluble raw material can then be extracted a second time with a solution that has a high concentration of acetone to obtain oils enriched in fatty acids and phytosterols.

### Example III

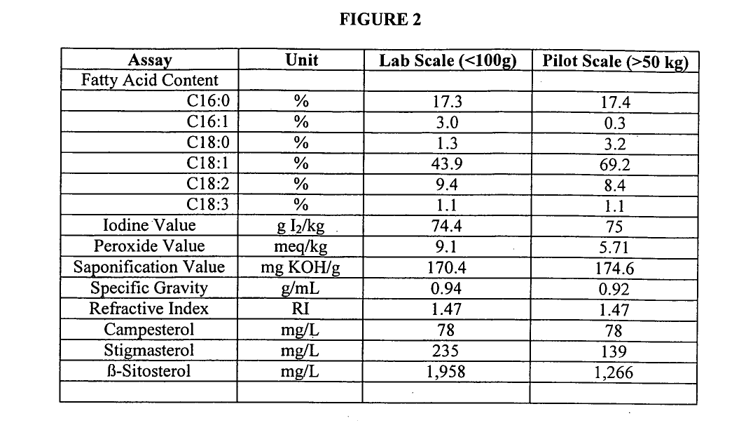

In a pilot-scale trail, açai oil was extracted with absolute ethanol and compared to the laboratory scale extraction for key chemical and physical characteristics of the oil (FIG. 2). Lipid profiles were conducted at a third party laboratory, with predominant percent fatty acids reported. Assays for iodine value give an indication of the degree of unsaturation, peroxide value as a measure of oxidation to the oil, saponification value for the average carbon chain-length of the oil, specific gravity for its weight per volume, and refractive index as indication of the type of fatty acids present. Four phytosterols were also identified in relatively high concentrations.

### Example IV

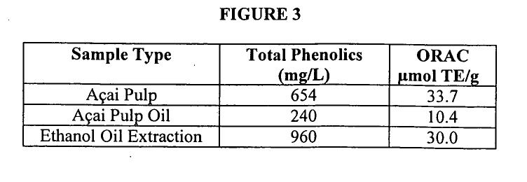

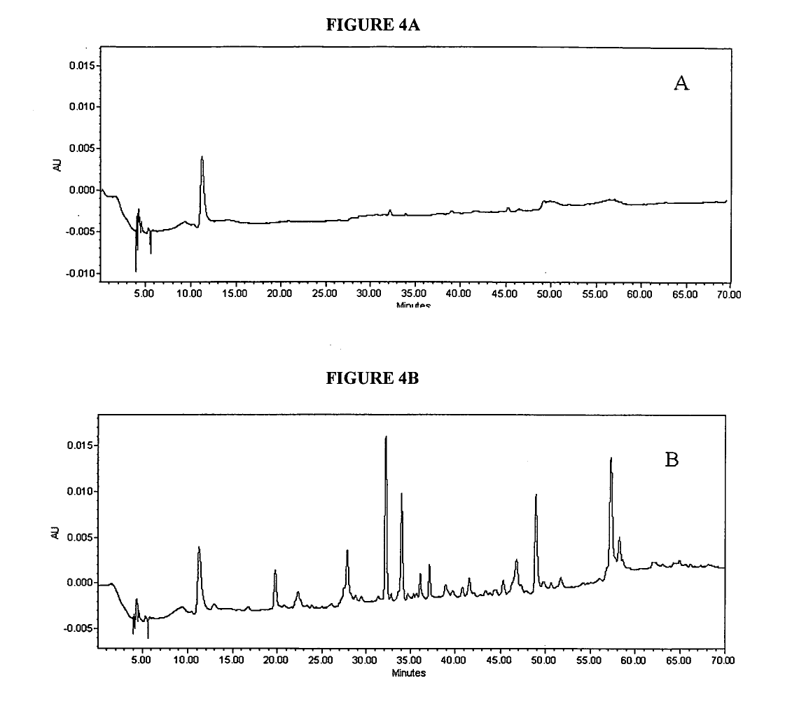

Oil physically removed from açai pulp by centrifugation was compared with ethanol-extracted oil from açai by-product as described in Example 1 was diluted with 50% methanol and 50% water to obtain sample for analysis. Individual polyphenolics were separated and characterized by HPLC using a Waters 2695 Alliance HPLC system, a Dionex Acclaim 120-C₁₈ column (250 mm×4.6 mm) with a C₁₈ guard column, and a Waters 996 PDA detector monitored at 280 nm. Separations were performed with a gradient mobile phase consisting of 68:30:2 (water:acetonitrile:acetic acid) mixed into water (2% acetic acid) on a reversed phase column at 0.8 mL/min. Polyphenols were identified by spectral interpretation, retention time, and comparison to authentic standards. By visually inspecting a chromatogram (FIG. 4B), it is evident that the processed oil has increased polyphenolics compounds. A side-by-side comparison for total polyphenolic and antioxidant capacity among açai pulp between oil derived from physical removal from the pulp following centrifugation and ethanol-extracted oil from açai processing by-product as described in Example 1 is shown in FIG. 3. From the data, it is apparent that the processed oil is higher in total soluble phenolics than the original açai pulp and the oil from that açai pulp. The isolated pulp oil contained little to no anthocyanins. Since anthocyanins are the predominant antioxidants in açai fruit, the high antioxidant capacity of the processed extracted oil demonstrates that it is indeed enhanced in antioxidant polyphenolics compared to the açai pulp and physically recovered açai oil from the pulp alone. Since non-anthocyanin polyphenolics account for less than one third of the total antioxidant capacity present in açai pulp, a significant enrichment in antioxidant agents is obtained in the ethanol-extracted oil from the açai fruit by-product described in Example 1.

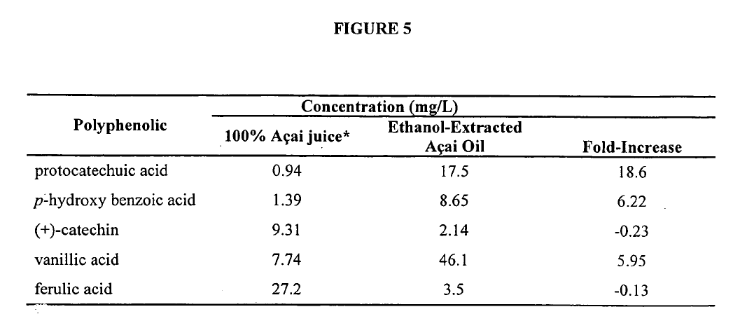

Compared to the non-anthocyanin containing açai juice (as described in Pacheco-Palencia et al., 2007), the ethanol-extracted oil is enhanced in compounds such as protocatechuic acid, p-hydroxybenzoic acid, and vanillic acid from 6 to 18-fold, while compounds such as (+)-catechin and ferulic acid were not (FIG. 5). This is believed to be due to selectivity and/or solubility of these compounds in the oil during the extraction and isolation procedure.

The stability of the antioxidants present in the ethanol-extracted oil from açai fruit by-product was also evaluated in a 10-Week shelf life at three storage temperatures (20°, 30°, and 40° C.) under a blanket of nitrogen. Compared to the results of the initial oil, there was only a minor change in the stability of the enhanced polyphenolics during storage under these extreme conditions. Additionally, no significant changes were observed to the free fatty acid or peroxide value of the oil during this storage, indicating excellent stability during storage. This also indicates that the bioactive compounds in the oils are not significantly altered by storage temperature or are protected by the oil matrix itself.

### Example V

Antioxidant constituents from açai juice, which could also be found at various concentrations in the açai oil, were tested in a cell culture model for cancer. The cancer cells utilized in the selected cell culture model were HL-60 cells, which are human leukemic carcinoma cells (blood cells). The model does not test the ability of açai to prevent leukemia, but rather the activity of açai isolated samples against a cancerous cell system. Compounds that show good activity against cancer cells in a model system are preferred.

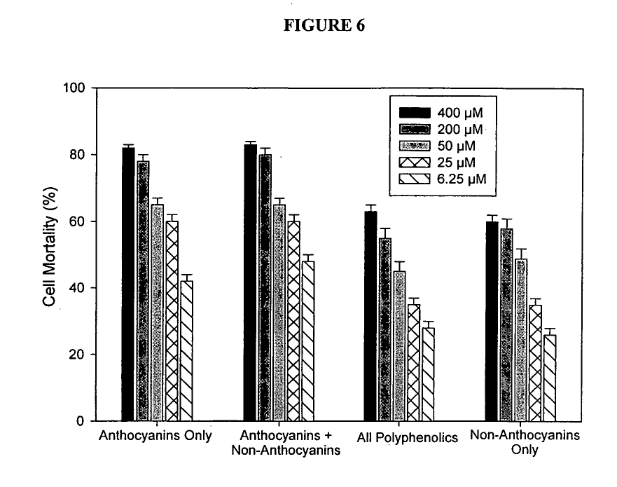

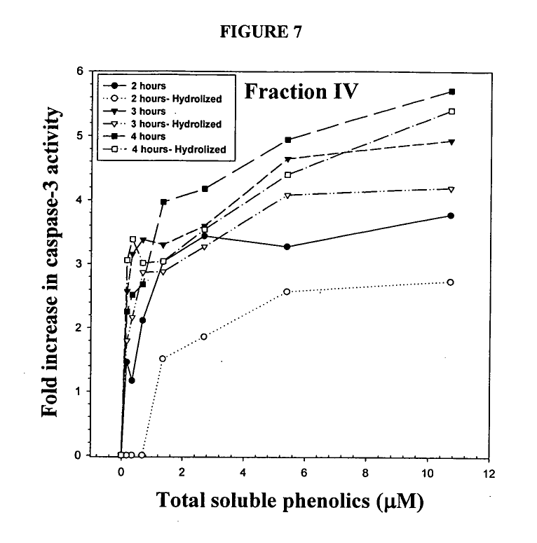

Based on an in vivo (human) absorption rate of 5% of the total compounds from açai, trials were conducted to determine efficacy in a dose-dependent response analogous to consuming from 4000 mL down to 62.5 mL of açai juice. These doses were adjusted for each chemical isolate obtained from açai including anthocyanins, non-anthocyanin polyphenolics, and their combination. Within the cell culture model, two assays were run, first for mitochondrial activity (FIG. 6) to assess whether the cells were alive or dead. The second assay tested for the enzyme caspase-3 (FIG. 7) that, once activated in a cell, is a terminal point beyond which the cell will not survive. Natural compounds such as polyphenolics may lead to the activation of caspase-3 in cancer cells, which then causes the cancer cells to die or cease mitochondrial activity.

Açai polyphenolics that contained anthocyanins, non-anthocyanin polyphenolics, or mixtures of these compounds demonstrated the highest antioxidant capacity in açai. These polyphenolics were tested as naturally found in açai (glycosides), but also tested in the absence of acylated sugar moieties in what is commonly referred to as their aglycone form. Each group of polyphenolics (glycosides and aglycones) had a significant impact on reducing the number of live cancer cells (higher percentage of cell mortality), where the higher concentrations killed more cancer cells than the lower concentrations. The lowest concentrations of glycosidic polyphenolics caused approximately 25%-48% cell mortality whereas the highest concentration caused approximately 64%-84% mortality. Hydrolyzed compounds generally caused an overall increase in cell mortality than the naturally occurring form of compounds. Polyphenolic fractions that contained anthocyanins were overall higher than non-anthocyanin polyphenolics in causing cell mortality. Results for cell mortality were compared to a standard of quercetin, a common polyphenolic compound found in many fruits and vegetables, and polyphenolics from açai compared favorably to the mortality induced by quercetin. Additionally, mortality was compared to camptotechin, a chemotherapy drug, and again açai compared favorably to this compound.

Likewise, the same comparisons were made for the activation of caspase-3. This enzyme is present in the cancer cells in an inactive form, and may be naturally activated as the cell ages or is induced into activation by the presence of compounds from açai. Thus, the timing of the enzyme action is critical, calling for testing of the enzyme over several time periods (2, 3 and 4 hours after introducing the polyphenolics to the cells) and in the glycosidic and aglycone forms. Quercetin was able to double the activity of caspase-3 at the optimal time and highest concentration whereas the maximal level attained for camptotechin was 7-fold higher activation. For the compounds isolated from açai, the non-anthocyanin polyphenolics induced 5.5-fold higher caspase-3 at the highest tested concentrations, with only small differences due to the glycoside versus aglycone forms. For the anthocyanins alone, activation was 9-fold higher at the highest concentrations for the aglycone form compared to a 6-fold increase for the glycosidic forms. When these compounds are combined, as found naturally in açai, the maximal effect was about 7-fold higher activation with little differences due to the glycosidic or aglycone forms.

The cell culture model demonstrated that açai contains compounds that possess significant activity to induce cancer cell mortality and induce an enzyme that most often causes the death of the cancer cell. Results tend to accentuate the activity of anthocyanins, the most prevalent polyphenolic in açai, but significant beneficial effects are also observed for the non-anthocyanin polyphenolics.

### Example VI

### Materials and Methods

Phytochemical Extracts. As described in Pacehco-Palencia et al., Journal of Agricultural and Food Chemistry 56, 3593-3600 (2008), incorporated herein by reference, frozen, pasteurized açai pulp was kindly donated by Bossa Nova Beverage Group (Los Angeles, Calif.) and shipped overnight to the Department of Nutrition and Food Science at Texas A&M University. Prior to polyphenolic isolation, açai pulp was clarified according to the procedure as described in Pacheco-Palencia et al., Food Research International 40, 620-628 (2007), hereby incorporated by reference, to remove insoluble solids. Phenolic acids and non-anthocyanin flavonoids were isolated from the clarified açai pulp by repeated liquid/liquid extraction with ethyl acetate (1:1 ratio). The upper ethyl acetate fraction was recovered, passed through a 5 cm bed of sodium sulfate to remove residual water, evaporated under vacuum (<40° C.), and re-dissolved in dimethyl sulfoxide (DMSO). Açai oil was extracted from a water insoluble filter cake that was commercially used to clarify açai pulp using a process as described herein from byproduct obtained during the clarification process. Polyphenolics' present in the resultant açai oil were extracted (three times) by the addition of methanol and water (80:20 v/v mixture) and centrifuged at 5000 g for 15 min. The methanolic extracts were then recovered, pooled, and concentrated under vacuum at <40° C. until complete solvent removal. The resultant extract was likewise reconstituted in DMSO to enhance solubility and used for further analyses. Polyphenolic isolates from both açai oil and açai pulp were standardized to a total soluble phenolic content of 1200 mg of gallic acid equiv (GAE)/L, corresponding to the total soluble phenolic content of single-strength açai oil and equivalent to a 300-fold concentrate of the açai pulp. Total soluble phenolics were analyzed by using the Folin-Ciocalteu assay as described in Singleton et al., American Journal of Enology and Viticulture 16, 144-153 (1965), incorporated herein by reference, and quantified against a gallic acid standard curve. All polyphenolic isolates were sterile-filtered prior to use in cell culture experiments and normalized to a final concentration of 0.1% DMSO when applied to the cells. A control with 0.1% DMSO was included in all assays.

Chemical Analyses. Polyphenolic isolates were analyzed by reversed phase HPLC with a Waters 2695 Alliance system (Waters Corp., Milford, Mass.), as described in Pacheco-Palencia et al., Food Chemistry 105, 28-35 (2007), hereby incorporated by reference. Identification and quantitation of polyphenolics were based on their spectral characteristics and retention time, as compared to authentic standards (Sigma Chemical Co., St. Louis, Mo.). Compound identities were further confirmed by mass spectrometric analyses, performed on a Thermo Finnigan LCQ Deca XP Max MSn ion trap mass spectrometer equipped with an ESI ion source (Thermo Fisher, San Jose, Calif.). Separations were conducted using the Phenomenex (Torrance, Calif.) Synergi 4 μHydro-RP 80A (2×150 mm; 4 μm; S/N=106273-106275) with a C18 guard column. Mobile phases consisted of 0.5% formic acid in water (phase A) and 0.5% formic acid in 50:50 methanol and acetonitrile (phase B) run at 0.25 mL/min. Polyphenolics were separated with a gradient elution program in which phase B changed from 5 to 30% in 5 minutes, from 30 to 65% in 70 minutes, and from 65 to 95% in 30 minutes and was held isocratic for 20 minutes. Ionization was conducted in the negative ion mode under the following conditions: sheath gas (N₂), 60 units/min; auxiliary gas (N₂), 5 units/min; spray voltage, 3.3 kV; capillary temperature, 250 ° C.; capillary voltage, 1.5 V; tube lens offset, 0 V. Total soluble phenolic contents were determined using the Folin-Ciocalteu assay as described in Singleton et al., American Journal of Enology and Viticulture 16, 144-153 (1965), incorporated herein by reference, quantified in gallic acid equivalents (GAE) as was used to quantify normalized concentrations between açai pulp and açai oil extracts. Antioxidant capacity was determined by the oxygen radical absorbance capacity assay using aBMG Labtech FLUOstar fluorescent microplate reader (485 nm excitation and 538 nm emission), as described in Talcott et al., Journal of Agricultural Food Chemistry 50, 3186-3192 (2002), incorporated herein by reference. Results were quantified in micromoles of Trolox equivalents per milliliter of extract.

Cell Proliferation. HT-29 human colon adenocarcinoma cells were obtained from American Type Culture Collection (ATCC, Manassas, Va.), cultured in Dulbecco's modified Eagle's medium (1×) (DMEM) containing 5% fetal bovine serum, 1% nonessential amino acids, 100 units/mL penicillin G, 100 μg/mL streptomycin, 1.25 μg/mL amphotericin B, and 10 mM sodium pyruvate (Gibco BRL Life Technology, Grand Island, N.Y.). Cells were incubated at 37° C. under 5% CO₂ and utilized between passages 10 and 20. Cells (5×10⁴ cells/well) were seeded into each well of a 12-well tissue culture plate. After a 24 hour incubation period, the growth medium was replaced with 1000 μL of medium containing different concentrations of standardized polyphenolic extracts (from 0.04 to 12 μg of GAE/mL). Following incubation for 48 hours, cell numbers were determined using a Beckman Coulter Particle Counter (Fullerton, Calif.). Cell numbers were expressed as a percentage of the 0.1% DMSO control. The extract concentration at which cell proliferation was inhibited by 50% (IC₅₀) was calculated by linear regression analyses on percentage cell inhibition as a ratio to the DMSO control.

Generation of Reactive Oxygen Species (ROS). The dichlorofluorescein (DCF) assay was performed according to Mertens-Talcott et al., Journal of Nutrition 135, 609-614 (2005), incorporated herein by reference. HT-29 human colon adenocarcinoma cells (5×10⁴/mL) were passed into 96-well plates and incubated for 24 hours. Cells were washed twice with PBS and preloaded with dichlorofluorescein diacetate (DCFH-DA) substrate by incubation with 10 μM DCFH-DA for 30 minutes at 37° C. Cells were subsequently washed and incubated with the standardized extract concentrations previously described. Fluorescence was determined after 30 minutes of incubation with polyphenolics using a BMG Labtech FLUOstar fluorescent microplate reader (485 nm excitation and 538 nm emission).

Transepithelial Transport Model. Caco-2 colon carcinoma cells were obtained from ATCC, cultured in DMEM (1×) high glucose containing 10% fetal bovine serum, 1% nonessential amino acids, 100 units/mL penicillin G, 100 μg/mL streptomycin, 1.25 μg/mL amphotericin B, and 10 mM sodium pyruvate. Cells were incubated at 37° C. and 5% CO₂ (chemicals were obtained from Sigma-Aldrich Co.). Cells between passages 10 and 20 were seeded in 12 mm transparent polyester cell culture insert well plates (Transwell, Coming Costar Corp., Cambridge, Mass.) at 1.0×10⁵ cells per insert with 0.5 mL of medium in the apical side and 1:5 mL of medium in the basolateral side. Cells were grown and differentiated to confluent monolayers for 21 days, as described in Hidalgo et al., Gastroenterology 96, 736-749 (1989), incorporated herein by reference. Transepithelial electrical resistance (TEER) values were monitored with an EndOhm Volt ohm-meter equipped with a STX-2 electrode (World Precision Instruments Inc., Sarasota, Fla.), and monolayers with TEER values >450 Ωcm² after correction for the resistance in control wells were used for transport experiments. TEER values were also obtained at the conclusion of transport experiments to ensure integrity of the monolayer. For transport studies, medium pH was adjusted to 6.0 on the apical side and 7.4 on the basolateral side using Hank's balanced salt solution (HBSS, Fischer Scientific, Pittsburgh, Pa.) containing 10 mM 2-(N-morpholino)ethanesulfonic acid solution (MES) and HBSS containing N-(2-hydroxyethyl) piperazine-N′-(2-ethanesulfonic acid) buffer solution (1 M) (HEPES) to create a pH gradient similar to the absorption sites in the small intestine environment (chemicals obtained from Gibco BRL Life Technology). Standardized polyphenolic extract solutions were diluted in HBSS (from 2.4 to 36 μg of GAE/mL) and loaded into the apical side of the cells. Transepithelial transport was followed over time, and sample aliquots (200 μL) were taken at 30, 60, and 120 minutes from the basolateral compartment, as described in De Castro et al., Journal of Pharmaceutical Sciences 96, 2808-2817 (2007), incorporated herein by reference. Samples were filtered prior to direct injection into the HPLC.

Statistical Analysis. Data from in vitro experiments were analyzed by one-way analysis of variance (ANOVA) using SPSS version 15.0 (SPSS Inc., Chicago, Ill.). Mean separations were conducted using posthoc Tukey-Kramer HSD (p<0.05) pairwise comparisons. Correlation and linear regression analyses were conducted using a significance level of 0.05.

### Results and Discussion

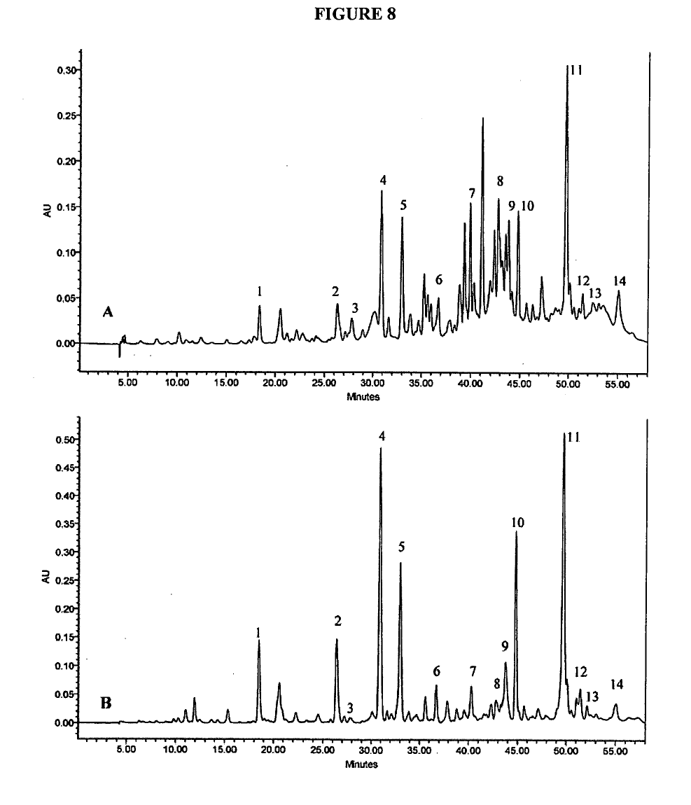

Chemical Analyses. Açai fruits are especially high in the anthocyanins cyanidin-3-rutinoside and cyanidin-3-glucoside, but in the manufacture of açai oil these compounds are not retained, whereas numerous phenolic acids and flavan-3-ols are solubilized in the açai oil following isolation from açai pulp clarification. Therefore, biological activities of phytochemical rich extracts from açai oil were compared to non-anthocyanin polyphenolic extracts from açai pulp and their respective chemical compositions contrasted under identical chromatographic conditions. Both açai oil and açai pulp contained similar phenolic acids, flavonoids, and procyanidins, but these compounds were present in different ratios in their respective matrices. Phenolic acids present were confirmed by LC-MS in negative ionization mode and included protocatechuic acid, p-hydroxybenzoic acid, vanillic acid, syringic acid, and ferulic acid along with the monomeric flavonols (+)-catechin and (−)-epicatechin. Each was identified on the basis of UV and mass spectrometric characteristics as compared to authentic standards. In addition, several procyanidin dimers and trimers were identified on the basis of their distinctive fragmentation patterns and spectral similarities to (+)-catechin and (−)-epicatechin. Procyanidin dimers were characterized by signals at m/z 577.1 and major fragments at m/z 425.0 and 289.2. Procyanidin trimers (m/z 865.1) were additionally characterized by predominant product ions at m/z 577.2, 425.0, and 289.2. Individual polyphenolic concentrations in açai pulp and açai oil extracts are presented herein (Table I). Similar polyphenolic profiles were observed in the açai oil extracts and non-anthocyanin polyphenolic extracts of açai pulp (FIG. 8); however, their absolute and relative ratios differed markedly, which was likely a contributing factor to their various bioactivities. Individual phenolic acid concentrations were up to 3.4-fold higher in açai oil extracts ranging from 540 to 1607 mg/L compared to açai pulp extracts at 159-577 mg/L. Ferulic acid was the one exception with nearly equivalent concentrations in açai oil and pulp. In contrast, flavanols such as (+)-catechin and (−)-epicatechin were abundant in pulp extracts but only sparingly present in extracts from açai oil. Likewise, procyanidin dimers in açai pulp extracts were twice those present in açai oil extracts, yet equivalent concentrations of procyanidin trimers were present in both extracts. Differences in composition between the açai extracts were attributed in part to extraction protocols, but primarily to the difference in the matrices (aqueous versus lipophilic) from which the polyphenolics were derived. Due to these differences in polyphenolics, the açai extracts were normalized to an equivalent concentration of total soluble phenolics (1200 mg of GAE/L) for their subsequent use in cell culture experiments. This concentration was comparable to concentrations originally present in the açai oil but a 300-fold concentration of polyphenolics present in açai pulp following anthocyanin removal. Resultant antioxidant capacity of the normalized extracts was 17.2 (0.16 μmol of Trolox equiv/mL for the açai pulp extract and 15.3 (0.11 μmol of Trolox equiv/mL for the açai oil extract due to differences in individual polyphenolics present.

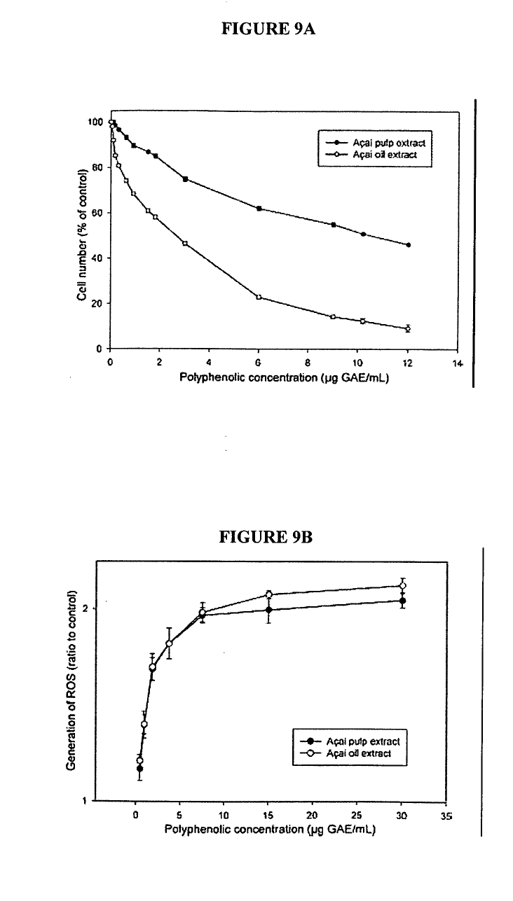

Cell Proliferation. The antiproliferative activities of açai pulp and açai oil extracts were evaluated in a cell culture model using HT-29 colon carcinoma cells. Total cell numbers were indicative of the proliferative activity of HT-29 cells and the cytotoxic effects of açai extracts. Both polyphenolic extracts caused significant (p<0.01) decreases in total cell numbers in a concentration-dependent manner (FIG. 9A). However, polyphenolic extracts from açai oil were more than twice as effective in reducing total cell numbers across all dilutions, with an IC₅₀ of 4.5 μg of GAE/mL compared to 10.2 μg of GAE/mL for açai pulp extracts. Previous studies have reported synergistic effects among polyphenolic mixtures in cell culture models as described in Mertens-Talcott et al., Cancer Letters 218, 141-151 (2005) and Chang et al., Toxicology and Applied Pharmacology 205, 201-212 (2005), both of which are incorporated by reference. Additionally, interactions among phenolic acids and flavanols may play an important role in the growth inhibitory action of açai pulp and açai oil extracts on cell proliferation observed in this study. Differences in the inhibitory effects by açai pulp and açai oil extracts may be due to their polyphenolic composition, because standardized açai oil extracts had 3.4-fold higher phenolic acid and 2-14-fold lower monomeric and dimeric flavanol concentrations than standardized extracts from pulp. Thus, results from this study suggest that cancer cell growth inhibition by these extracts is associated with phenolic acids and their interactions among polyphenolic components. Studies on cranberries in HT-29 cell models have suggested non-additive interactions involving flavanol derivatives as described in Seeram et al., Journal of Agricultural and Food Chemistry 52, 2512-2517 (2004), and Ferguson et al., Journal of Nutrition 134, 1529-1535 (2004), both of which are hereby incorporated by reference, whereas potent growth inhibitory effects of cloudberry, bilberry, raspberry, black currant, strawberry, and lingonberry extracts were attributed to synergistic interactions among non-anthocyanin polyphenolics, including phenolic acids and flavanols, that enhanced expression of p21WAF1 as an inducer of cell cycle arrest and apoptosis as described in Wu et al., Journal of Agricultural and Food Chemistry 55, 1156-1163 (2007), incorporated herein by reference. Cell growth inhibition by polyphenolic-rich açai extracts was related to total soluble phenolic concentrations originally present in açai pulp (4 μg of GAE/mL) and in the açai oil (1200 μg of GAE/mL). Therefore, 10 μg of GAE/mL of açai pulp extract resulted in 50% reduction of cell proliferation, whereas comparable inhibition was observed for only 3 μg of GAE/mL of the açai oil extract (FIG. 9A). Therefore, 10 μg of GAE of açai pulp extract, which corresponded to the phenolic equivalent present in 2.5 mL of açai pulp, and 3 μg GAE of açai oil extract, which corresponded to the equivalent of 2.5 μL of açai oil, were both equivalent in terms of their inhibitory effects on cell proliferation. These observations suggest that the polyphenolic enriched açai oil, obtained from a water-insoluble filter cake used commercially to clarify açai pulp, was >3 times more effective than the non-anthocyanin polyphenolics isolated from açai pulp for inhibition of colon carcinoma cell proliferation when present at equal concentrations. Although in vitro antioxidant activity of polyphenolic compounds has been associated with potential health benefits as described in Hou et al., Current Molecular Medicine 3, 149-159 (2003), hereby incorporated by reference, results from this study suggest that inhibition of cancer cell proliferation in vitro by phenolic acids and flavonoids in açai extracts may be independent of their antioxidant activity. Potential mechanisms for this chemopreventive activity may be due to inhibition of carcinogen formation, immunoregulation, disruption of cell cycle, enzyme regulation, and enhancement of DNA repair levels as described in Nichenametla et al., Critical Reviews in Food Science and Nutrition 46, 161-183 (2006), hereby incorporated by reference.

Generation of ROS. The generation of ROS was evaluated by the DCF assay. Both açai pulp and açai oil extracts induced a significant, concentration-dependent increase in the generation of ROS (FIG. 9B). However, unlike the anti-proliferative effect, no significant differences between açai pulp or açai oil extracts were detected at any of the concentrations tested. Both extracts induced the generation of ROS at concentrations between 0.4 and 30 μg of GAE/mL. Their inductive effects were more pronounced at lower concentrations (<5 μg of GAE/mL) and decreased markedly above 7.5 μg of GAE/mL. Similarly, low polyphenolic concentrations (<5 μg of GAE/mL) of açai pulp or açai oil extracts were effective at increasing average ROS generation rates. Decreased generation of ROS by higher polyphenolic extract concentrations may be attributable to the presence of sufficient polyphenolics in the model to reduce the generation of ROS through their antioxidant potential; however, due to the complex nature of these reactions, this influence can only be hypothesized. The observed differences between açai pulp and açai oil extracts in the inhibition of HT-29 cell proliferation were not explained by their similarities in the rate and extent of generation of ROS, suggesting that induction of ROS was not the primary mechanism responsible.

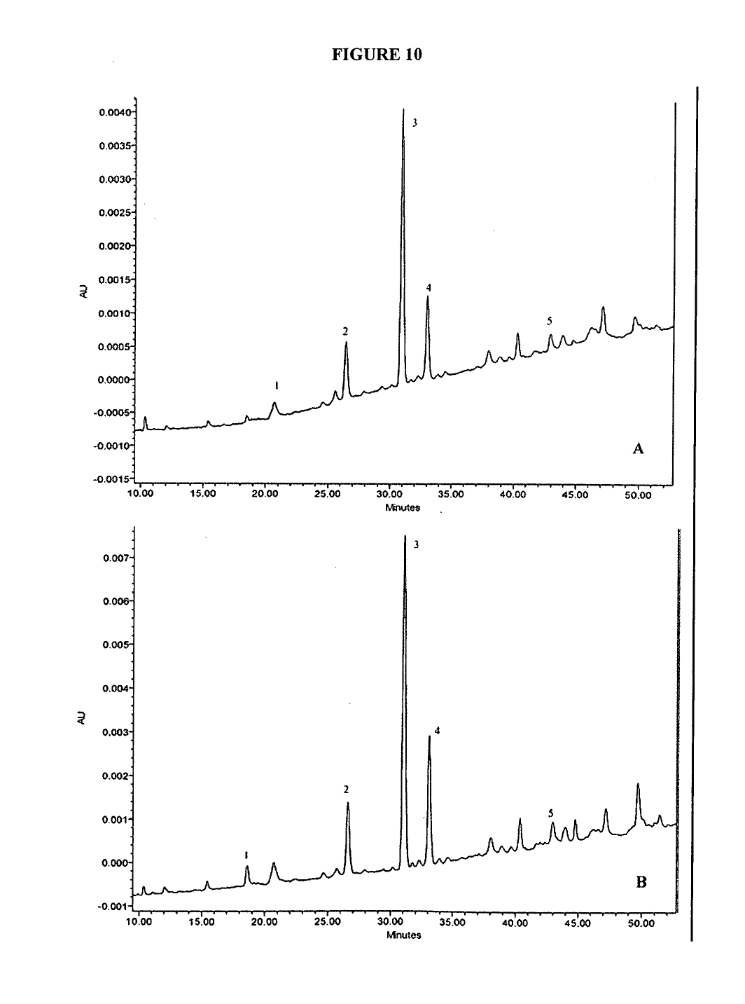

Transepithelial Transport Model. Transport of non-arithocyanin polyphenolics from açai pulp and açai oil extracts was also evaluated using Caco-2 cell monolayers as an in vitrointestinal absorption model. Caco-2 cells are the most extensively characterized and functional in vitro model in the field of drug absorption and permeability as described in Balimane et al., Journal of Pharmacological and Toxicological Methods 44, 301-312 (2000), incorporated herein by reference, and were previously used to evaluate intestinal absorption and transport of various phenolic acids as described in Konishi et al., Journal of Agricultural and Food Chemistry 51, 7296-7302 (2003), and Kobayashi et al., Journal of Agricultural and Food Chemistry 48, 5618-5623 (2000); flavonoids as described in Walgren et al., Biochemical Pharmacology 55, 1721-1727 (1998), and Vaidyanathan et al., Pharmaceutical Research 18, 1420-1425 (2001); and procyanidins as described in Deprez et al., Antioxidants and Redox Signaling 3, 957-967 (2001), all of which are hereby incorporated by reference. Transport of polyphenolics across the Caco-2 monolayers was studied in the apical to basolateral direction. Extracts were loaded into the apical side of the cell monolayers, and individual polyphenolic concentrations appearing in the basolateral side were evaluated over time by HPLC, after incubation for 0.5, 1, and 2 hours. Analytical HPLC chromatograms of polyphenolics present in the basolateral solutions after incubation for 2 hours are presented in FIG. 10. Phenolic acids such as p-hydroxybenzoic, vanillic, syringic, and ferulic acids were transported from the apical to the basolateral side of Caco-2 cell monolayers, along with monomeric flavanols such as (+)-catechin and (−)-epicatechin, when present in complex polyphenolic mixtures. Average transport rates (μg/mL·h) of non-anthocyanin polyphenolics from açai pulp and açai oil extracts adjusted to different concentrations (μg of GAE/mL), from the apical to the basolateral side of Caco-2 cell monolayers were given in time (Table II). Individual polyphenolic transport rates (0.02-10.4 μg/mL·h) increased in a concentration-dependent manner (24-360 μg of GAE/mL) for both extracts; however, absolute phenolic acid transport rates were significantly higher (p<0.05) for açai oil extracts than for their açai pulp counterparts at equivalent concentrations. The opposite was observed for (+)-catechin and (−)-epicatechin monomers, as indicative of the polyphenolic profiles of açai pulp and açai oil extracts. Variations in individual polyphenolic transport rates were negligible at low phenolic extract concentrations (approximately 24 μg/mL·h) with the exception of vanillic and syringic acid, for which transport efficiencies were enhanced at higher extract concentrations. Whereas the presence of methyl groups increased polyphenolic transport rates, the presence of polar hydroxyl groups was associated with increased membrane retention from hydrogen-bond formation with polar functional groups on lipids at the lipid/water interface as described in Ollila et al., Archives of Biochemistry and Biophysics 399, 103-108 (2002), hereby incorporated by reference. In contrast, flavanols are primarily transported via paracellular diffusion as described in Journal of Agricultural and Food Chemistry 51, 7296-7302 (2003), hereby incorporated by reference; therefore, higher (+)-catechin and (−)-epicatechin transport rates in cells loaded with açai pulp polyphenolic extracts may be due to higher initial concentrations in açai pulp extracts compared to açai oil extracts. Relative transport of açai polyphenolics from açai pulp and açai oil extracts following incubation for 2 hours is summarized in Table III. Transport efficiencies were expressed as the percentage of the initial polyphenolic concentration (loaded in the apical side) detected on the basolateral side of Caco-2 cell monolayers following incubation for 2 hours. In contrast to previous observations on polyphenolic transport rates, relative transport efficiencies of individual polyphenolics did not vary as a function of initial polyphenolic concentrations and showed signs of saturation above polyphenolic concentrations of about 30 μg of GAE. Transport efficiencies for p-hydroxybenzoic (approximately 2.0%), vanillic (approximately 1.0%), and syringic (approximately 0.6%) acids were not affected (p<0.05) by initial polyphenolic concentrations in açai pulp extracts nor were efficiencies for ferulic acid (approximately 0.1%) and (+)-catechin (approximately 0.1%) in açai oil extracts. Similar transport efficiencies were observed for ferulic acid, (+)-catechin, and (−)-epicatechin at açai pulp extract concentrations above 60 μg/mL. Transport of phenolic acids such as p-hydroxybenzoic, vanillic, and syringic from açai oil extracts increased proportionally to the amount originally loaded into the apical compartment, up to a concentration of 240 μg/mL, after which no further changes in transport were observed. Results from this study suggest that non-anthocyanin polyphenolic extracts from açai pulp and from a polyphenolic-enriched açai oil obtained from a commercial açai pulp clarification process are sources of biologically active phenolic acids and flavan-3-ols. Polyphenolic-rich extracts from açai pulp and açai oil significantly inhibited cell proliferation and increased the generation of ROS in a concentration-dependent manner. Despite the generation of ROS from these compounds in açai fruit, further mechanisms are likely responsible for the potent cytotoxicity of açai oil extracts on HT-29 colon cancer cells. Transepithelial transport of these phenolic compounds was also evaluated in Caco-2 monolayers as a model for intestinal absorption and demonstrated significant transport from the apical to the basolateral side of the monolayer. These data demonstrate that the chemical composition has an appreciable influence on the cell proliferation and absorption properties of phenolic acids and flavonoids from açai fruit and açai oil. Results from this study provide further evidence of the anti-proliferative properties of açai fruit polyphenolics in cultured cancer cells and offer new insights on their absorption.

### Example VII

### Materials and Methods

Materials and Processing. The food-grade açai oil used in these trials was isolated using hydroalcoholic solvents from a water-insoluble filter cake commercially used to clarify açai pulp in the manufacture of açai juice as described herein to recover both triacylglycerides and polar phenolic compounds. The filter cake was obtained from the Bossa Nova Beverage Group (Los Angeles, Calif.) and was held frozen (−20° C.) until the açai oil isolation procedure on a pilot scale. Solvent removal was accomplished using a falling film evaporator, and the resultant açai oil was essentially free of water and solvent. This initial açai oil was designated as “high phenolics” açai oil, since the oil was naturally enriched in phenolics trapped in the filter cake. A modified version of this açai oil was prepared by repeatedly (five times) extracting the high phenolics açai oil with water (1:5 ratio) to remove water-soluble phenolic compounds and then extracted with 100% hexane to facilitate isolation of predominantly the triacylglycerols. Hexane was removed from the açai oil under reduced pressure at <40 ° C., resulting in açai oil that contained phenolics at a concentration <5% of the original and was designated as a “low phenolics” açai oil. A third modification of the original açai oil was prepared as a 50:50 (v/v) blend of the first two açai oils and designated as “intermediate phenolics” açai oil. Equal amounts of each açai oil (5 mL) were loaded into screw-cap glass test tubes in triplicate, and the headspace was flushed with nitrogen, and the samples were stored at 20, 30, and 40° C. in the dark for 10 weeks. Individual tubes were removed from storage periodically and held at −20° C. until analysis. Additionally, a short-term evaluation of the thermal stability of phenolics in the high phenolic açai oil was evaluated by loading 2 mL into a screw-cap glass test tube and heating to an internal temperature of 150 and 170° C. for 0, 5, 10, and 20 minutes using peanut oil as the heating medium. Samples were immediately cooled by immersion in cold water. Following each stability trail, açai oil samples (100 mg) were extracted with 4 mL of a 1:1 (v/v) hexane:methanol mixture until the oil was fully dissolved. After dissolution, a known volume of 0.1 M aqueous citric acid buffer at pH 3.0 was added to form a bilayer from which the lower, hydrophilic phase was retained for subsequent chemical analyses. Phytochemical analyses of the enriched açai oil were also compared to a non-anthocyanin phenolic extract obtained from açai fruit. A fruit pulp was obtained from the frozen skins of açai fruit by cold maceration with water and was subsequently clarified by removing lipids and insoluble solids with centrifugation and filtration. To obtain chemically similar phenolic extracts as was obtained from the açai oil, the açai fruit extract was extensively liquid/liquid extracted with ethyl acetate (1:1) to isolate non-anthocyanin phenolics. The solvent was passed through a 5 cm bed of sodium sulfate to remove residual water and evaporated under reduced pressure at <40° C., and the isolate was re-dissolved in a known volume of 0.1 M citric acid buffer (pH 3.5) for subsequent analyses.

Chemical Analyses. Major phenolic compounds present in açai oil were analyzed by reversed-phase high-performance liquid chromatography (HPLC) with a Waters 2695 Alliance system (Waters Corp., Milford, Mass.) over 60 minutes, according to previously described chromatographic conditions as described in Pacheco-Palencia et al., Food Research International 40, 620-628 (2007), incorporated herein by reference. Phenolics were identified and quantified based on their spectral characteristics and retention times, as compared to authentic standards (Sigma Chemical Co., St. Louis, Mo.). Further structural information was obtained by mass spectrometric analyses, performed on a Thermo Finnigan LCQ Deca XP Max MSn ion trap mass spectrometer equipped with an ESI ion source (ThermoFisher, San Jose, Calif.). Separations were conducted using the Phenomenex (Torrace, Calif.) Synergi 4 μm Hydro-RP 80A (2 mm×150 mm; 4 μm; SIN=106273-106275) with a C18 guard column. Mobile phases consisted of 0.5% formic acid in water (phase A) and 0.5% formic acid in 50:50 methanol:acetonitrile (phase B) run at 0.25 mL/minute. Phenolics were separated with a gradient elution program in which phase B changed from 5 to 30% in 5 minutes, from 30 to 65% in 70 minutes, and from 65 to 95% in 30 minutes and was held isocratic for 20 minutes. Electrospray ionization was conducted in the negative ion mode under the following conditions: sheath gas (N₂), 60 units/minute; auxiliary gas (N₂), 5 units/minute; spray voltage, 3.3 kV; capillary temperature, 250° C.; capillary voltage, 1.5 V; and tube lens offset, 0 V. Total soluble phenolics, measuring the metal reduction capacity of açai oil extractions, was analyzed by the Folin-Ciocalteu assay as described in Singleton et al., American Journal of Enology and Viticulture 16, 144-153 (1965), incorporated herein by reference, and quantified in gallic acid equivalents (GAE). The antioxidant capacity was determined using the oxygen radical absorbance capacity assay as described in Talcott et al., Journal of Agricultural and Food Chemistry 50, 3186-3192 (2002), incorporated herein by reference, using fluorescein as the fluorescent probe on a BMG Labtech FLUOstar fluorescent microplate reader (485 nm excitation and 538 nm emission). Results were quantified in 82 mol Trolox equivalents per milliliter of açai oil. The overall oxidative stability of the açai oils was determined by measuring free fatty acids and peroxide value according to AOAC official methods as provided for in Horwitz et al., Official Methods of Analysis of AOAC International (17^{th} ed.), AOAC International, Gaithersburg, Md. (2002), hereby incorporated by reference.

Statistical Analyses. Data from each chemical analysis were analyzed by one-way analysis of variance using SPSS version 15.0 (SPSS Inc., Chicago, Ill.). Mean separations were conducted using Tukey-Kramer HSD (p<0.05) as a post-hoc analysis. Linear and nonparametric correlations among chemical analyses were obtained, and linear regression analyses were conducted using a significance level of 0.05.

### Results and Discussion

The phenolic-enriched açai oil from the byproduct of açai fruit clarification was physically characterized by its dark green color, viscous nature, and a distinctive aroma reminiscent of açai fruit pulp. Further chemical tests were conducted by an independent contract laboratory (Medallion Laboratories, Minneapolis, Minn.), including specific gravity (0.9247 g/cm³), refractive index (1.4685), iodine value (75.0 I₂/100 g oil), peroxide value (5.71 meq/kg oil), and fatty acid composition (17.4% palmitic acid, 0.3% palmitoleic acid, 3.2% stearic acid, 69.2% oleic acid, 8.4% linoleic acid, and 1.1% linolenic acid).

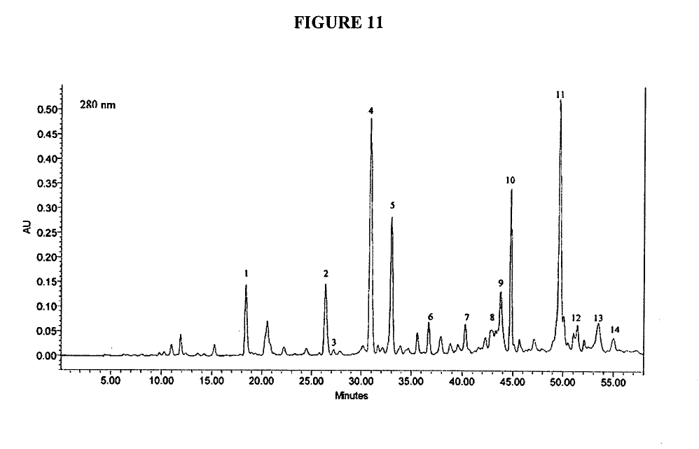

Phenolic Characterization. The phenolic compounds (FIG. 11) present in açai oil were co-extracted from the water-insoluble residues of açai pulp processing and were identified based on their spectrophotometric characteristics and mass spectra and quantified against authentic standards when available. Phenolic acids such as vanillic acid (1616±94 mg/L), syringic acid (1073±62 mg/L), p-hydroxybenzoic acid (892±52 mg/L), protocatechuic acid (629±36 mg/L), and ferulic acid (101±5.9 mg/L) were predominantly present in the açai oil, while (+)-catechin (66.7±4.8 mg/L), four B type procyanidin dimers [1085.6±121.3 mg (+)-catechin equiv/L], and four procyanidin trimers [2016.2±53.2 mg (+)-catechin equiv/L] were also detected in high concentrations (Tables IV and V). In addition, five compounds exhibiting typical flavonoid spectral characteristics were detected at trace concentrations, from 0.58 to 4.21 mg rutin equiv/kg in açai oil. Previous studies on phenolics in açai pulp as provided for in Gallori et al., Chromatographia 59, 739-743 (2004), and Lichtenhaler et al., International Journal of Food Science and Nutrition 56, 53-64 (2005), both of which are incorporated by reference; seeds as described in Rodrigues et al., Journal of Agricultural and Food Chemistry 54, 4162-4167 (2006), hereby incorporated by reference; and freeze-dried açai fruit as provided for in Schauss et al., Journal of Agricultural and Food Chemistry 54, 8598-8603 (2006), incorporated herein by reference, reported the presence of phenolic acids such as vanillic, syringic, p-hydroxybenzoic, protocatechuic, and ferulic acid, as well as (+)-catechin, (−)-epicatechin, and B type procyanidins, from dimers to high molecular weight polymers that were not characterized. In this study, structural information was obtained by means of mass spectrometric analyses that confirmed the identities of the phenolics previously identified by HPLC and provided additional information on the identity and degree of polymerization of procyanidin dimers and trimers (Table IV). Both the catechin-like UV spectroscopic properties and the characteristic signals at m/z 577.1 ([M−H]−) indicated the presence of procyanidin dimers, while signals at m/z 865.1 ([M−H]−) were attributed to procyanidin trimers. Previous LC-ESI-MSn studies on proanthocyanidins agreed that the m/z 577.1 ion is indicative of B-type procyanidin dimers as described in Friedrich et al., European Food Research and Technology 211, 56-64 (2000), incorporated herein by reference, while fragmentation to the m/z 425.0 ion was characteristic of the product obtained from retro-Diels-Alder reaction of ring C and subsequent elimination of ring B from the flavan-3-ol as provided for in Friedrich et al., European Food Research Technology 211, 56-64 (2000), and Gu et al., Journal of Mass Spectrometry 38, 1272-1280 (2003), both of which are incorporated herein by reference. Finally, m/z 289.2 and 287.1 fragments, likely from cleavage of the inter-flavanoid bond as described in Friedrich et al., European Food Research Technology 211, 56-64 (2000), and Pati et al., Journal of Mass Spectrometry 41, 861-871 (2006), both of which are hereby incorporated by reference, suggested that these procyanidin dimers consisted of two (+)-catechin or (−)-epicatechin units, although no differentiation between isomers was possible. Similarly, procyanidin trimers (m/z 865.1) were characterized by a predominant product ion at m/z 577.2, likely corresponding to a dimeric fragment ion from cleavage of interflavanoid linkages, which has been recognized as the most important fragmentation mechanism in proanthocyanidin trimers as provided for in Friedrich et al., European Food Research Technology 211, 56-64 (2000), incorporated herein by reference. Further fragmentation of m/z 577.2 occurred in a similar manner as in the previously described procyanidin dimers. Açai oil extracts used in these trials did not contain anthocyanins and were therefore compared with non-anthocyanin phenolics present in clarified açai pulp by chemically contrasting contents and concentrations under identical chromatographic conditions (Table V). Similar phenolic profiles were observed in the açai oil extracts and non-anthocyanin phenolic extracts of açai pulp; yet, their concentrations differed markedly. Because of an enhanced extraction and concentration of compounds from water-insoluble residues from açai pulp clarification, the individual phenolics were significantly higher in açai oil than in açai pulp by 12.6-469.4 times. Dimeric and trimeric procyanidins were predominant in both açai oil extracts (3102±127 mg/L) and açai pulp extracts (17.2±2.2 mg/L) and accounted for over 40% of the total phenolics present. Similarly, phenolic acids such as vanillic, syringic, protocatechuic, p-hydroxybenzoic, and ferulic acids were predominant in both the açai oil extracts (101-1616 mg/L) and the açai pulp extracts (1.1-5.5 mg/L). However, their respective abundances differed significantly (p<0.05) between products, and with the exception of ferulic acid, phenolic acids were appreciably enhanced in the açai oil (Table V). Although both (+)-catechin and (−)-epicatechin were found in açai pulp (1.1 and 5.3 mg/kg, respectively), only (+)-catechin (66.7±4.8 mg/kg) was present in açai oil extracts. The similarities between the phenolic profiles of açai pulp and açai oil extracts suggest that these phenolics have the ability to be extracted from açai byproduct sources (e.g., insoluble solids from açai pulp) as free or bound compounds and deposited to a nonpolar lipid phase, which serves to appreciably enhance phenolic concentrations in the açai oil when compared to the fruit pulp from which they were derived.

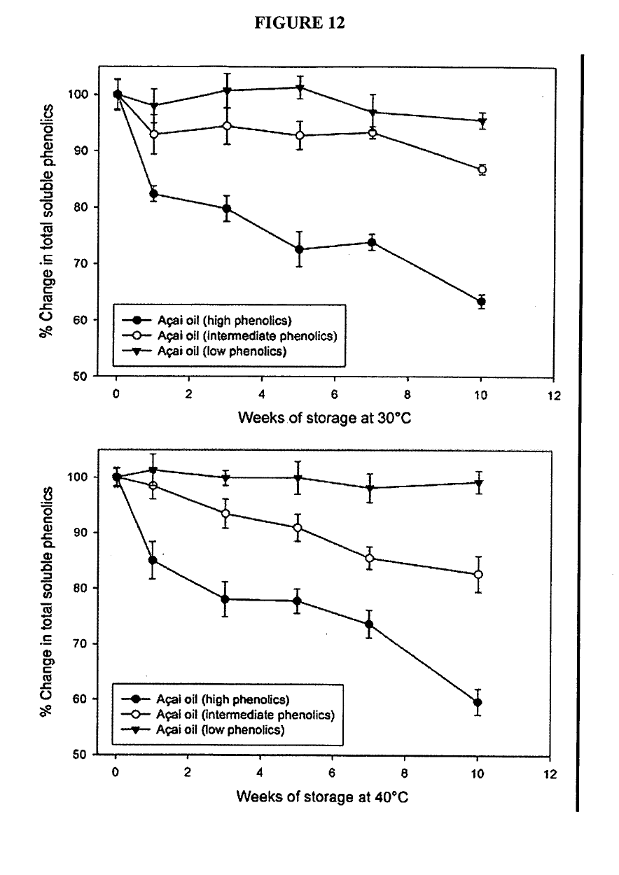

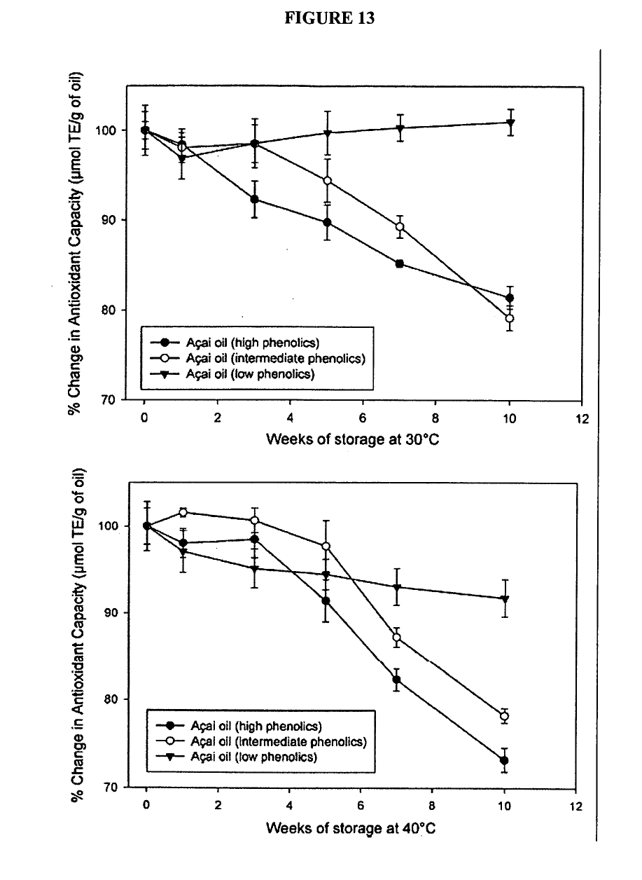

Phenolic Storage Stability. The influence of naturally occurring phenolics on the phytochemical stability of extracted açai oil during storage was evaluated by monitoring changes to individual phenolics, total soluble phenolic content, and antioxidant capacity of polar isolates in açai oil stored at 20, 30, or 40° C. for 10 weeks. Initial phenolic concentrations in the açai oil were adjusted to three concentration levels containing high, intermediate, and low phenolic concentrations by diluting original açai oil extracts with açai oil whose phenolics were removed by aqueous extraction. The açai oil containing the lowest phenolic concentration contained <5% of the original açai oil's concentration, whereas the intermediate oil was 50% of the original (Table VI). Individual phenolic concentrations were monitored periodically during storage, and no significant differences (p<0.05) were found between high and intermediate phenolic oils (Table VII), suggesting that phenolic losses were independent of initial phenolic contents. Moreover, no significant changes in (+)-catechin, protocatechuic acid, p-hydroxybenzoic acid, vanillic acid, or syringic acid concentrations were detected after 10 weeks of storage at 20 or 30 ° C., and only minor changes (<10%) were observed following storage at 40° C., indicating excellent storage stability of these compounds even under adverse handling conditions. Storage effects were more pronounced for ferulic acid and procyanidin dimers and trimers, as concentrations decreased by 9.3 and 23.2%, respectively, when stored at 20° C., by 20.3 and 39.1% when stored at 30° C., and by 33.1 and 73.4% when stored at 40 ° C. Because of the high stability of flavanol monomers during storage and earlier reports on the stable nature of procyanidins as described in Rios et al., American Journal of Clinical Nutrition 76, 1106-1110 (2002), incorporated herein by reference, it was hypothesized that decreased concentrations of procyanidin dimers and trimers during storage might be attributed to açai oil matrix effects on procyanidin extraction efficiency, such as the formation of complexes between procyanidins and proteins or other oil-soluble components over time. Evidence for complexation that decreased solubility of procyanidins was found upon further analyses of the açai oil following the initial phenolic extraction. Because phenolic acids and monomeric flavonoid concentrations remained constant during storage, total soluble phenolic contents were used as a potential indicator of the presence of residual procyanidins in the phenolic-extracted açai oil. Results indicated that total soluble phenolic contents in the phenolic-extracted açai oil were 64.7±3.9 mg GAEIL and thus reflected the presence of oil-bound procyanidins in the oil that was enhanced during oil storage. Açai oil extracts were additionally evaluated for soluble phenolic contents, as a measure of total reducing capacity, and for changes in antioxidant capacity throughout the storage period. A statistically significant (p<0.01) correlation was found between total soluble phenolic content and antioxidant capacity by both linear (r=0.94) and nonparametric (p=0.92) methods during storage at 20, 30, and 40° C. The high phenolic oil had an initial antioxidant capacity of 21.5±1.7 μM TE, Trolox equivalents/mL, and a total soluble phenolic content of 1252±42 mg GAE/L, whereas the intermediate phenolic oils were about half the SE levels at 14.3±1.2 μM TE/mL and 695±28 mg GAE/L and low phenolic oils at 4.8±0.3 μM TE/mL and 192±8.3 mg GAE/L, respectively. Total soluble phenolics expressed as GAE were found at appreciably lower concentrations than the sum of individual phenolic concentrations (Table VII) and were attributable to the high concentration of hydroxybenzoic acids in the açai oil that was previously shown to exhibit poor reducing capacity and radical scavenging activity as described in Kim et al., Critical Reviews in Food Science and Nutrition 44, 253-273 (2004), and Rice-Evans et al., Free Radical Biology and Medicine 20, 933-956 (1996), both of which are hereby incorporated by reference. The antioxidant activity of phenolic acids was shown to be dependent on the number of hydroxy substitutions on their aromatic ring as described in Kim et al., Critical Reviews in Food Science and Nutrition 44, 253-273 (2004), incorporated herein by reference; however, the electron-withdrawing properties of the carboxyl group in benzoic acids have a negative effect on hydrogen-donating abilities of hydroxybenzoic acids as described in Rice-Evans et al., Free Radical Biology and Medicine 20, 933-956 (1996), incorporated herein by reference. During storage, the total soluble phenolic content of the high phenolic oil decreased by 36.1-40.3% when stored at 20, 30, or 40° C. (FIG. 12), corresponding to a 18.6-26.8% decrease in antioxidant capacity (FIG. 13). Both the intermediate and the low phenolic oils experienced significantly (p<0.05) smaller losses for these attributes during storage ranging from minor losses (<10%) in the low phenolic oil at all storage temperatures and as high as 21.8% in the intermediate phenolic oil at 40° C. Linear regression analyses of antioxidant capacity and total soluble phenolic contents during storage further confirmed a significant (p<0.01) influence of açai oil phenolic concentration on retention of both total soluble phenolics and antioxidant capacity during storage, but no significant effect was attributed to storage temperature. Such differences might be attributed, at least partially, to previously observed differences in procyanidin concentrations (Table VI), which were more pronounced in high and intermediate phenolic oils.

Oil Storage Stability. The oxidative stability of açai oil extracts adjusted to three different phenolic concentrations was further assessed by monitoring changes in free fatty acid (% oleic acid) and peroxide values (mequiv/kg) following storage. Free fatty acid (<0.1%) and peroxide values (<10 mequiv/kg) were unchanged prior to and after storage of the açai oils at each temperature, indicating that lipids did not experience significant oxidative changes (data not shown) after 10 weeks of storage at 20, 30, or 40° C.

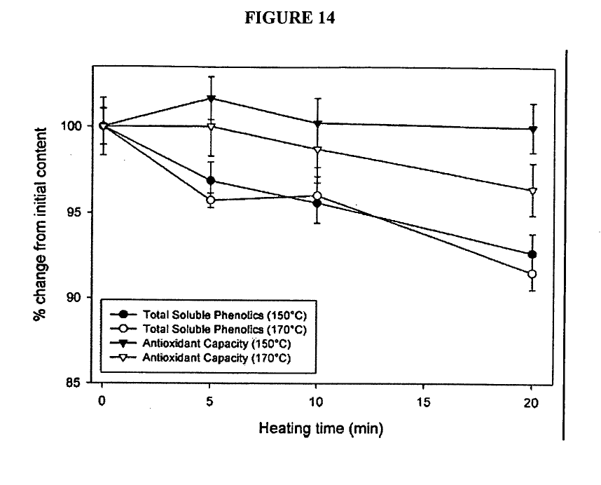

Phenolic Thermal Stability. The short-term, high-temperature storage stability of phenolics in açai oil extracts was evaluated by monitoring changes in total soluble phenolic contents, antioxidant capacity, and individual phenolic concentrations following heating of the high phenolic oil to a temperature of 150 or 170° C. and holding for 5, 10, and 20 minutes. This short-term trial was to simulate cooking effects on the açai oil and to determine if the phytochemical composition and stability would degrade under moderate to high temperatures. However, even under the most extreme time and temperature combination, no changes in the physical nature of the açai oil (color alterations and viscosity changes) were observed, and no significant (p<0.05) changes to individual phenolic concentrations were detected during this evaluation. Minor variations in overall antioxidant capacity (<5%) and soluble phenolic contents (<10%) were observed under these same conditions (FIG. 14), with slightly greater losses observed at 170° C. as compared to 150° C. Therefore, the extracted açai oil demonstrated excellent thermal stability for the phenolics present and indicated its potential for culinary applications involving moderate exposure times to high temperatures.

Conclusion. The phytochemical composition of açai oil extracts from water-insoluble residues of açai pulp processing was characterized and found to be appreciably enhanced in nonanthocyanin phenolics such as phenolic acids and procyanidins. Individual phenolic contents were not significantly altered by long-term storage at temperatures up to 40° C. for 10 weeks or by short-term heating at temperatures up to 170° C. for 20 minutes, indicating good stability of these compounds and their antioxidant properties. Because of its high phenolic content, storage stability, and unique sensory characteristics, açai oil is a promising new alternative to traditional oils for food, supplements, and cosmetic applications.

We claim:

1. An isolated non-naturally occurring oil composition comprising:
greater than 50% by weight unsaturated fatty acids;
greater than 10% by weight saturated fatty acids; and
greater than 0.1% by weight of polyphenolics and;
greater than 0.1% of phytosterols.

2. The oil composition of claim 1, wherein said oil composition comprises less than 0.5% by weight of polyphenolics.

3. The oil of claim 1 wherein said polyphenolics comprise:
protocatechuic acid (3,4-dihydroxybenzoic acid);
procyanidin C1 (Epicatechin-(4beta-->8)epicatechin-(4beta-->8)epicatechin;
p-hydroxybenzoic acid;
(+)-catechin;
vanillic acid;
(-)-epicatechin;
proanthocyanidin A2 ((+)-Epicatechin-(4beta-8,2beta-O-7)-epicatechin); and
ferulic acid.

4. The oil of claim 1, comprising greater than 10 mg per liter of protocatechuic acid (3,4-dihydroxybenzoic acid).

5. The oil of claim 1, comprising greater than 10 mg per liter of vanillic acid.

6. The oil of claim 1, wherein said phytosterols comprises beta-sitosterol, stigmasterol, and campesterol.

7. The composition of claim 1, wherein the oil composition is obtained from an açai fruit.

8. The oil of claim 1, wherein said saturated and unsaturated fatty acids do not contain trans fatty acids.

9. The oil of claim 1, wherein said composition is 60%-90% by weight unsaturated fatty acids, 10-20% by weight saturated fatty acids, 0.1%-0.5% polyphenolics, and 0.1%-0.5% phytosterols.

10. The oil of claim 1, wherein said composition comprises 60%-80% by weight monounsaturated fatty acids, 10%-20% by weight polyunsaturated fatty acids, and by weight 10%-20% saturated fatty acids.

11. An isolated non-naturally occurring oil composition having a chromatographic profile through a reverse-phase column substantially similar to FIG. 4B.

12. A method of isolating an açai fruit oil comprising:
a) providing a composition comprising açai mesocarp,
b) mixing said composition with an extraction solution comprising one or more chemical moieties selected from the group consisting of ketones and alcohols such that a second solution comprising a set of insoluble components is formed;
c) filtering said second solution to separate said set of insoluble components providing a third solution; and
d) isolating an açai fruit oil by removing volatile components from said third solution.

13. The method of claim 12, wherein said extraction solution comprises between 10% to 50% acetone by volume and 50% to 90% ethanol by volume.

15. The method of claim 12, wherein said extraction solution comprises about 57% ethanol, 3% water, and about 40% acetone.

16. The method of claim 12, wherein said _{K}ai oil contains a total polyphenolics concentration of at least 1,000 mg per liter of oil.

17. The method of claim 12, wherein said açai oil contains a total phytosterol concentration of at least 1,000 mg per liter of oil.

18. The method of claim 12, wherein said composition comprises açai fruit exocarp.

19. The method of claim 12, wherein said açai fruit oil has a moisture content of less than 0.5%.

20. A method of isolating an açai fruit oil comprising:
a) providing a composition comprising açai mesocarp having a first viscosity;
b) mixing said composition and a carbohydrase under conditions such that a first solution comprising a first set of insoluble components is formed with a second viscosity that is less that said first viscosity;
c) separating said first set of insoluble components from said first solution;
d) mixing said insoluble components with an extraction solution comprising an alcohol such that a second solution comprising a second set of insoluble components is formed;
e) filtering said second solution to separate said second set of insoluble components providing a third solution; and
f) isolating an açai fruit oil by removing volatile components from said third solution.

22. The method of claim 20, wherein said extraction solution comprises between 30% to 50% acetone by volume and 50% to 70% ethanol by volume.

23. The method of claim 20, wherein said extraction solution comprises about 57% ethanol, 3% water, and about 40% acetone by volume.

24. A composition comprising a component comprising açai mesocarp and an extraction solution comprising one or more chemical moieties selected from the group consisting of ketones and alcohols.

25. The composition of claim 24, wherein said extraction solution comprises between 10% to 50% acetone by volume and 50% to 90% ethanol by volume.

26. The composition of claim 24, wherein said extraction solution comprises between 30% to 50% acetone by volume and 50% to 70% ethanol by volume.

27. The composition of claim 24, wherein said extraction solution comprises about 57% ethanol, 3% water, and about 40% acetone.

28. A method of isolating an açai fruit oil comprising:
a) providing a composition comprising açai mesocarp,
b) mixing said composition with a first extraction solution comprising alcohol such that a second solution comprising a first set of insoluble components is formed;
c) separating said first set of insoluble components from said second solution;
d) mixing said first set of insoluble components with a second extraction solution comprising ketone such that a third solution comprising a second set of insoluble
f) isolating an açai fruit oil by removing volatile components from said fourth solution.

29. The method of claim 28, wherein said first extraction solution comprises between 99% to 60% ethanol by volume and 1% to 40% acetone by volume.

30. The method of claim 28, wherein said second extraction solution comprises between 99% to 60% acetone by volume and 1% to 40% ethanol by volume.

31. The method of claim 28, wherein said first extraction solution comprises between 99% to 60% acetone by volume and 1% to 40% ethanol by volume.

32. The method of claim 28, wherein said second extraction solution comprises between 99% to 60% ethanol by volume and 1% to 40% acetone by volume.

33. The composition of claim 28, wherein said açai fruit oil comprises a total phytosterol concentration of at least 1,000 mg per liter of oil.

34. The composition of claim 28, wherein said açai fruit oil enriched in phytosterols comprises a beta-sitosterol concentration of at least 1,000 mg per liter of oil.

35. A cosmetic product comprising the composition of claim 1.

36. A nutritional supplement comprising the composition of claim 1.

37. A pharmaceutical comprising the composition of claim 1.
greater than 50% by weight unsaturated or polyunsaturated fatty acids;
greater than 10% by weight saturated fatty acids; and
greater than 0.1% by weight of polyphenolics; and
greater than 0.1% by weight of phytosterols.

40. The composition of claim 39, wherein said oil is obtained from an açai fruit.

41. The method of claim 39, further comprising the step of diluting the composition prior to orally administering said composition.
